(19) United States
(12) Patent Application Publication
(10) Pub. No.: US 2025/0361483 A1
(43) Pub. Date: Nov. 27, 2025

(54) Systems, Methods, and Compositions for Selecting or Isolating Cells

(71) Applicant: NATIONAL RESILIENCE, INC., San Diego, CA (US)

(72) Inventors: Sebastian Bohm, San Diego, CA (US); Dwight Baker, San Diego, CA (US); Martin Kolnik, San Diego, CA (US); Mazen Karaman, San Diego, CA (US); G. Anthony Reina, San Diego, CA (US)

(21) Appl. No.: 19/123,686

(22) PCT Filed: Oct. 24, 2023
(86) PCT No.: PCT/US2023/077579

§ 371 Date: Apr. 23, 2025

Related U.S. Application Data
(60) Provisional application No. 63/380,687, filed on Oct. 24, 2022.
(60) Provisional application No. 63/485,853, filed on Feb. 17, 2023.

(51) Int. Cl. C12N 5/0783; B01D 37/00; B03C 1/00; G01N 33/543

(52) U.S. Cl. CPC C12N 5/0636; B01D 37/00; B03C 1/00; G01N 33/543

(57) ABSTRACT

Systems, methods, and compositions can be used for separating, isolating and/or selecting cells. The methods can utilize beads and/or matrices that bind cells. The beads and/or matrices can be dissolvable. The disclosed systems, methods, and compositions can include magnetic particles and/or buoyant components. The disclosed systems, methods, and compositions can implement size selection.

### CROSS-REFERENCE TO RELATED APPLICATIONS

This application claims priority to U.S. Provisional Patent Application No. 63/380,687, filed on Oct. 24, 2022, and U.S. Provisional Patent Application No. 63/485,853, filed on Feb. 17, 2023, each of which is entirely incorporated herein by reference.

### BACKGROUND

Cells can be used to generate cell therapies. Cell therapy can be used to treat diseases in individuals by providing cells to a subject. Cell therapies can be effective at treating multiple diseases or disorders, such as cancer, hematologic condition, immune disorders, neurological disorder. Additionally, examination of cells from an individual can be used for diagnosis of the diseases.

### SUMMARY

In an aspect, the present disclosure provides a method of selecting cells, the method comprising: a) providing a plurality of cells and a plurality of beads, wherein at least one bead of the plurality of beads comprises a binding agent capable of binding a subset of the plurality of cells; b) subjecting the plurality of cells and plurality of beads to conditions to allow the plurality of beads to bind the subset of the plurality of cells, thereby generating (i) at least one bead-cell complex, and (ii) at least one unbound cell, wherein the at least one bead-cell complex is a larger size compared to the at least one unbound cell; c) subjecting the at least one bead-cell complex and the at least one unbound cell to a size separation, thereby separating the at least one bead-cell complex from the at least one unbound cell. In some embodiments, the size separation comprises use of a filter. In some embodiments, a size of pores of the filter is less than a size of a bead-cell complex and greater than a size of the at least one unbound cell. In some embodiments, the size separation comprises at least one of inertial focusing and deterministic lateral displacement. In some embodiments, the plurality of beads comprises buoyant beads. In some embodiments, the size separation comprises use of a filter and pushing the at least a subset of the unbound cells through the filter. In some embodiments, the method further comprises, subsequent to c), collecting separated unbound cells. In some embodiments, the method further comprises, subsequent to c), collecting at least one separated bead-cell complex. In some embodiments, the method further comprises, subsequent to the collecting, subjecting the at least one separated bead-cell complex to at least one release condition, thereby releasing the subset of the plurality of cells from the plurality of beads. In some embodiments, the release condition comprises a high salt concentration solution. In some embodiments, the plurality of cells are immersed in a medium, and wherein the release condition comprises a change in the pH of the medium. In some embodiments, the change in pH comprises an increase of pH. In some embodiments, the change in pH comprises a decrease of pH. In some embodiments, the medium is a separation buffer. In some embodiments, the pH is changed from physiological to a pH value above about 8. In some embodiments, the release condition comprises a biotin solution. In some embodiments, the biotin solution comprises a desthiobiotin-based binding agent. In some embodiments, the biotin solution comprises a recombinant biotin having a binding affinity lower than native biotin. In some embodiments, the binding agent and the bead are linked via a linker. In some embodiments, the linker comprises a biotin and streptavidin. In some embodiments, the linker comprises a covalent linker. In some embodiments, the linker is generated by (i) a bead comprising a first reactive group and (ii) a binding agent comprising a second reactive group and reacting the first reactive group with the second reactive group to form the linker. In some embodiments, the linker is generated by (i) a bead comprising a first binding member and (ii) a binding agent comprising a second binding member and reacting the first binding member with the second binding member to form the linker. In some embodiments, the first binding member comprises biotin and the second binding member comprises streptavidin. In some embodiments, the first binding member comprises streptavidin and the second binding member comprises biotin.

In an aspect, the present disclosure provides a method of selecting cells, the method comprising: a) providing in a reaction chamber (i) a plurality of polymer precursors and (ii) a plurality of binding agents capable of binding a subset of a plurality of cells; b) subjecting the reaction chamber to polymerization conditions to generate, in the reaction chamber, a 3-dimensional (3D) matrix comprising the plurality of binding agents; c) introducing the plurality of cells into the reaction chamber to allow the plurality of binding agents to bind the subset of the plurality of cells, thereby generating (i) at least one bound cell, and (ii) at least one unbound cells; d) washing the 3D matrix to remove the at least one unbound cells; and e) subjecting the 3D matrix to a dissolving reagent to dissolve the 3D matrix, thereby releasing the subset of the plurality of cells.

In some embodiments, the reaction chamber is in a microfluidic device. In some embodiments, b) comprises contacting the polymer precursors with a polymerization reagent. In some embodiments, the polymer precursors comprise, for example, alginate or other dissolvable hydrogel, and the polymerization agent comprises calcium ions or salts. In some embodiments, the plurality of binding agents comprises antibodies or derivatives thereof. In some embodiments, the antibodies or derivatives thereof comprise scFvs, nanobodies, or Fab domains. In some embodiments, the dissolving reagent comprises citrate, EDTA, and/or alginase, for example.

In an aspect, the present disclosure provides a method of selecting cells, the method comprising: a) providing a plurality of magnetic beads in a reaction chamber, wherein at least one magnetic bead of the plurality of magnetic beads comprises a binding agent capable of binding a subset of a plurality of cells; b) introducing the plurality of cells into the reaction chamber to allow the plurality of magnetic beads to bind the subset of the plurality of cells, thereby generating (i) at least one magnetic bead-cell complex and (ii) at least one unbound cell; c) subjecting (i) the at least one magnetic bead-cell complex and (ii) the at least one unbound cells to a magnetic field to separate the at least one magnetic bead-cell complex from the at least one unbound cell; and d) subjecting the at least one magnetic bead-cell complex to a dissolving reagent to dissolve the at least one magnetic beads, thereby releasing the subset of a plurality of cells. In some embodiments, the at least one magnetic bead comprises a plurality of paramagnetic nanoparticles, wherein the plurality of paramagnetic nanoparticles are released during d) to yield released paramagnetic nanoparticles. In some embodiments, the method further comprises harvesting the released paramagnetic nanoparticles with a magnet. In some embodiments, the plurality of magnetic beads comprises alginate. In some embodiments, the dissolving agent comprises at least one of citric acid, EDTA, and alginase. In some embodiments, the binding agents comprises an antibody or derivatives thereof. In some embodiments, the antibody of derivatives thereof comprises antibody fragments. In some embodiments, the antibodies or derivatives thereof comprise scFvs, nanobodies, or Fab domains.

In an aspect, the present disclosure provides a method of manufacture of dissolvable magnetic beads comprising a) loading a microfluidic droplet generator with (i) a liquid alginate solution comprising magnetic nanoparticles, and (ii) a mineral oil, into a microchannel; and b) subjecting the liquid alginate solution to crosslinking, thereby forming dissolvable magnetic beads. In some embodiment, the method further comprises loading the microfluidic droplet generator with a binding agent in a), wherein the dissolvable magnetic beads comprise the binding agent.

In an aspect, the present disclosure provides a method of manufacture of dissolvable magnetic beads comprising a) spray drying a solution of alginate mixed with magnetic nanoparticles; and b) reconstituting the spray-dried alginate-magnetic nanoparticles in a buffer solution. In some embodiments, the solution further comprises a plurality of binding agents, and wherein the dissolvable magnetic beads comprise the plurality of binding agents. In some embodiments, the plurality of binding agents comprises an antibody or a derivative thereof.

In another aspect, the present disclosure provides a method of selecting cells, the method comprising: (a) providing a plurality of beads in a reaction chamber, wherein at least one bead of the plurality of beads comprises a binding agent capable of binding a subset of a plurality of cells; (b) introducing the plurality of cells into the reaction chamber to allow the plurality of beads to bind the subset of the plurality of cells, thereby generating (i) at least one bead-cell complexes, and (ii) at least one unbound cell; (c) subjecting (i) at least one bead-cell complex, and (ii) at least one unbound cell to a separation to separate (i) the plurality of bead-cell complexes from (ii) the plurality of unbound cells; and (d) subjecting the bead-cell complexes to a dissolving reagent to dissolve the plurality of beads, thereby releasing the subset of a plurality of cells. In some embodiments, the binding agent comprises an antibody. In some embodiments, the plurality of beads comprise alginate. In some embodiments, the dissolving reagent comprises citrate. In some embodiments, the size selection comprises use of a filter.

In another aspect, the present disclosure provides a method of selecting cells, the method comprising: a) providing, in a chamber, a plurality of cells and a plurality of buoyant beads, wherein at least one buoyant bead of the plurality of buoyant beads comprises a binding agent capable of binding a subset of the plurality of cells, wherein the chamber comprises: a plunger configured to pressurize liquid in the chamber, and the chamber is connected to an input channel and an output channel that is separated from the chamber with a filter. b) subjecting the plurality of cells and plurality of buoyant beads to conditions to allow the plurality of buoyant beads to bind the subset of the plurality of cells, thereby generating i) at least one buoyant bead-cell complex, and ii) a plurality of unbound cells, wherein the at least one buoyant bead-cell complex is a larger size than a pore of the filter and at least a subset of the plurality of unbound cells is smaller than the pore of the filter; c) initiating the plunger to push the at least one buoyant bead-cell complex and the plurality of unbound cells towards the filter, wherein the at least a subset of the plurality of unbound cells are able to traverse through the filter and the at least one buoyant bead-cell complex is unable to traverse through the filter. d) subjecting the at least one buoyant bead-cell complex to a release condition, thereby releasing the subset of the plurality of cells from the buoyant beads. e) initiating the plunger to push the subset of plurality of cells towards the filter, wherein the subset of plurality of cells are able to traverse through the filter and exit the chamber. In some embodiments, the plunger comprises modulating the plunger between push and pull conditions. In some embodiments, the filter comprises a sieve. In some embodiments, the binding agent comprises an antibody. In some embodiments, a buoyant bead of the plurality of the buoyant bead comprises streptavidin. In some embodiments, the binding agent comprises a biotin and is linked to the buoyant bead via a biotin-streptavidin interaction. In some embodiments, the release condition comprises flowing a solution comprising biotin. In some embodiments, the release condition comprises flowing a solution comprising a high salt concentration. In some embodiments, release condition comprises flowing a solution comprising a pH lower than the pH of a solution in reaction chamber. In some embodiments, the release condition comprises flowing a solution comprising a pH lower than the pH of a solution in reaction chamber. In some embodiments, the binding agent can bind a CD3, CD4, or CD8 protein, for example. In some embodiments, the binding agent can bind T-cells.

In another aspect, the present disclosure provides a system for selecting cells, the system comprising: a) a chamber comprising a plurality of buoyant beads; b) an input channel fluidically connected to the chamber; c) an output channel fluidically connected to the chamber; d) a filter disposed at an entrance of the output channel from the chamber, and e) a plunger disposed in the chamber and configured to apply pressure to a fluid in the chamber and push the fluid through the filter into the output channel.

In some embodiments, a buoyant bead of the plurality of buoyant beads comprises a binding agent. In some embodiments, the binding agent comprises an antibody. In some embodiments, a buoyant bead of the plurality of the buoyant bead comprises streptavidin. In some embodiments, the binding agent comprises a biotin and is linked to the buoyant bead via a biotin-streptavidin interaction. In some embodiments, the binding agent can bind a CD3, CD4, or CD8 protein. In some embodiments, the binding agent can bind T-cells.

In another aspect, the present disclosure provides a non-transitory computer readable medium comprising instructions that, when executed by a computer processor, cause the computer processor to perform methods described in this disclosure.

In another aspect, the present disclosure provides non-transitory computer readable medium comprising instructions that, when executed by a computer processor, cause the computer processor to operate systems described in this disclosure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a schematic of an example method of size selection.

FIG. 2. shows an example of an etched surface for use with size selection.

FIG. 3 shows a schematic of a DLD device.

FIG. 4 shows a schematic of an example method with dissolvable beads.

FIGS. 5A-C shows schematics of magnetic beads and their use.

FIG. 6A shows a schematic of a bead. FIG. 6B shows a schematic for an example method of use for beads.

FIG. 7 shows a computer control system that is programmed or otherwise configured to implement methods provided herein.

FIG. 8 shows a schematic of an example method for rapid immunophenotyping of cell biomarkers by inertial sorting.

FIG. 9 shows a schematic of an example method for separating target T cells from other white blood cells using a separation particle that can bind to a surface antigen on a T cell.

FIGS. 10A-10B show components of a separation method using magnetic nanoparticles. FIG. 10A shows an example of an open gradient magnet. FIG. 10B shows target cells eluted by resuspension in a testing tube.

FIG. 11 shows results of polystyrene bead size-based inertial separation. Unlabeled T cells are enriched in Outlet 1, while labeled T cells (e.g., T cell bound to bead) are recovered in Outlet 2.

FIGS. 12A-12B shows results of liquid microbubble using buoyancy separation. FIG. 12A shows testing tubes at stages of cell separation. The left tube shows cells prior to separation and the right tube shows results after separation and flushing of non-target cells. FIG. 12B shows results before the pressure pulse (left) and after the pressure pulse (right). Arrows designate cells.

FIG. 13 shows results of cell separation using magnetic microparticles coated with dissolvable polymer matrix. Arrows show free-floating particles (labeled [G]) and T cells bound to particles.

FIG. 14 shows flow cytometry plots of a PBMC sample stained with CD45, CD3, and CD4 antibodies, and 7-AAD viability dye to identify viable CD4+ T cells.

FIG. 15 shows purified CD4+ T cells immediately following separation with magnetic microparticles coated with dissolvable polymer matrix (e.g., beads).

FIG. 16 shows purified CD4+ T cells following dissolution of the beads.

FIG. 17 shows a schematic of the experimental setup to assess the purity and recovery of the separated CD4+ T cells. Results were compared to a negative control bead (e.g., no RPA-T4 IgG antibody conjugated to the polymer matrix shell of the magnetic bead).

FIG. 18 shows the purity results following cell separation. “pos” cells indicate cells after first magnetic separation and still bound to the beads. “posTneg” cells indicate cells captured after dissolution of the beads and removal of the supernatant. “posTpos” cells indicate the fraction of cells resuspended in pellet. All groups show greater purity levels than negative control beads.

FIG. 19 shows recovery results following cell separation as a fraction of CD4+ T cells in the original sample. Labeling is similar to that in FIG. 18. All purified cell groups showed higher recovery levels compared to those of negative control beads.

### DETAILED DESCRIPTION

Provided herein are systems, methods, and compositions for isolation and selection of cells. The systems and methods can be used to isolate or select cells, and the isolated or selected cells can be used for diagnosis or treatment (e.g., of a disorder or disease) in a subject. The cells can be isolated or selected to identify the presence of a cell. The systems, methods, and compositions allow for an improved generation of cell-based therapies, detection of diseased cells, or other methods or assays that utilize isolated cells. For example, cells that can be precursors for autologous cell therapies can be initially isolated using the methods, compositions, and systems described herein.

As used herein, the term “antibody” refers to an immunoglobulin (Ig), polypeptide, or a protein having a binding domain which is, or is homologous to, an antigen-binding domain. The term further includes “antigen-binding fragments” and other interchangeable terms for similar binding fragments as described below.

Antibodies and antigen-binding fragments herein can be partly or wholly synthetically produced. An antibody or antigen-binding fragment can be a polypeptide or protein having a binding domain which can be, or can be homologous to, an antigen binding domain. In one instance, an antibody or an antigen-binding fragment can be produced in an appropriate in vivo animal model and then isolated and/or purified. It would be understood that the antibodies and antigen-binding fragment herein can be modified as described herein or as known in the art.

Antibodies useful in the present invention encompass, but are not limited to, monoclonal antibodies, polyclonal antibodies, antibody fragments (e.g., Fab, Fab′, F(ab′)2, Fv, Fc, scFv, scFv-Fc, Fab-Fc, scFv-zipper, scFab, crossFab, camelids (VHH), etc.), chimeric antibodies, bispecific antibodies, multispecific antibodies, heteroconjugate antibodies, single chain (ScFv), mutants thereof, fusion proteins comprising an antibody portion (e.g., a domain antibody), humanized antibodies, human antibodies, and any other modified configuration of the immunoglobulin molecule that comprises an antigen recognition site of the required specificity, including glycosylation variants of antibodies, amino acid sequence variants of antibodies, and covalently modified antibodies.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. Amino acid residues of CDRs and framework regions are as described herein for the provided sequences.

A “constant region” of an antibody refers to the constant region of the antibody light chain or the constant region of the antibody heavy chain, either alone or in combination.

“Epitope” refers to that portion of an antigen or other macromolecule capable of forming a binding interaction with the variable region binding pocket of an antibody. Such binding interactions can be manifested as an intermolecular contact with one or more amino acid residues of one or more CDRs. Antigen binding can involve, for example, a CDR3 or a CDR3 pair or, in some cases, interactions of up to all six CDRs of the VH and VL chains. An epitope can be a linear peptide sequence (“continuous”) or can be composed of noncontiguous amino acid sequences (“conformational” or “discontinuous”). An antibody can recognize one or more amino acid sequences; therefore, an epitope can define more than one distinct amino acid sequence. Epitopes recognized by antibodies can be determined by peptide mapping and sequence analysis techniques well known to one of skill in the art. Binding interactions are manifested as intermolecular contacts between an epitope on an antigen and one or more amino acid residues of a CDR. An epitope herein can refer to an amino acid sequence on a receptor binding domain or a spike domain.

An antibody can selectively bind to a target if it binds with greater affinity, avidity, more readily, and/or with greater duration than it binds to other substances. For example, an antibody or antigen-binding fragment that selectively binds to a CD3 is an antibody or antigen-binding fragment that binds this epitope with greater affinity, avidity, more readily, and/or with greater duration than it binds to CD4 or CD8. For example, if the binding agent is an antibody binding a CD3 protein, it can be referred to as an anti-CD3 antibody and the CD3 protein may be referred to as an antigen.

The term “Fc region” is used to define a C-terminal region of an immunoglobulin heavy chain. The “Fc region” may be a native sequence Fc region or a variant Fc region. The Fc region of an immunoglobulin generally comprises two constant domains, CH2 and CH3.

The methods provided can comprise the use of solid supports for separation, isolation, and/or selection of cells. A solid support (e.g., a bead) can be used to bind a cell. The binding of cells to the solid supports can be leveraged to isolate or select for cells by using differences in the sizes of the objects. The resulting cell-bead complex can be a larger size than the size of a cell alone. As the bead-cell complexes are a different size from the bead without bound cells or a cell alone, size-based separation, isolation, and/or selection that can resolve the size differences can be used. Size-based separation, isolation, and/or selection techniques can be used to select bead-cell complexes over beads or cells alone. Size based separation, isolation, and/or selection strategies can include the use of semi-permeable membranes or filters that comprise pores or orifices of certain sizes. The pores can be large enough that a cell alone can pass through, but a bead and/or bead-cell complex may be too large to pass through. In this way, the pore size can allow for the selection or isolation of cells.

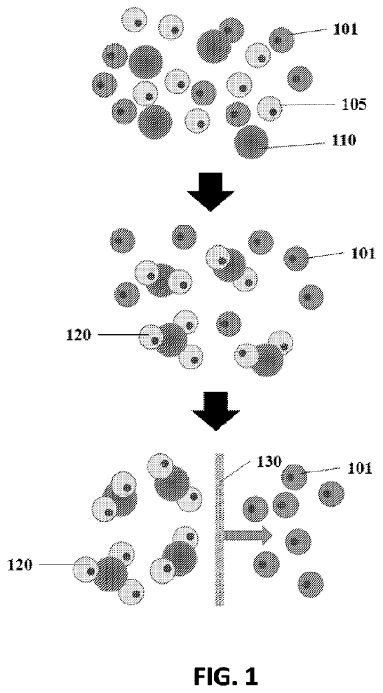

FIG. 1 shows an example of size-based selection to isolate cells. A plurality of cells 101, 105 and beads 110 that can bind cells are allowed to interact in a liquid (e.g., buffer, media, or another solution). Positive or negative selection can be used (i.e., beads can bind targeted cells or non-targeted cells). Negative selection can use beads that bind to cells that are not targeted (e.g., cells to be discarded) whereas the cells that are to be isolated and/or selected do not bind to the bead. Positive selection, on the other hand, can use beads that bind to the cells to be isolated and/or selected, whereas the non-targeted cells do not bind to the bead. FIG. 1 shows a selection mechanism, where cells 105 can bind to beads 110 to form bead-cell complexes 120. The liquid can then flow through a separation filter 130 that has pores that are smaller than the beads 110 (and bead-cell complexes 120). The beads 110 and bead-cell complexes 120 are unable to pass through the pores, whereas unbound cells 101 can pass through. When negative selection is used, the cells that have passed through the pores can be harvested for further processing, for example. The negative selection scheme can provide the advantage of extracting cells that do not comprise a label or a binding agent bound to the cell. These cells can then be directly added to downstream processing steps without the need to remove the label or binding agent. This can reduce the number of the steps needed for separation, isolation, and/or selection and can also avoid the addition of other reagent to de-bead the cells, which can cause adverse effects or reduce cell quality.

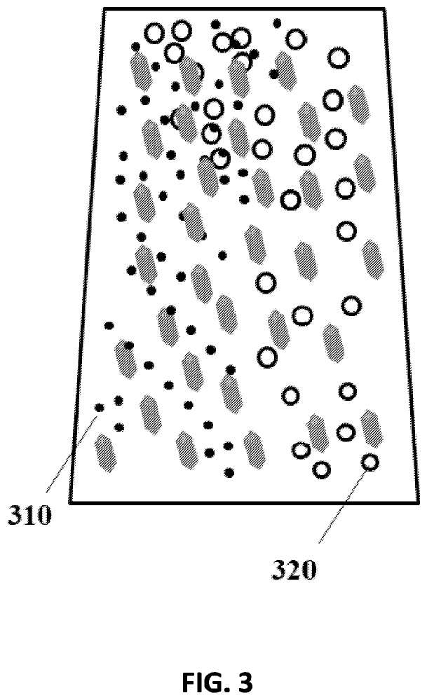

Size-based selection can also be performed using a Deterministic Lateral Displacement (DLD) device (see, e.g., FIG. 3). DLDs include several micropillars that are placed such to direct particles of different sizes to take different paths. For example, smaller particles 310 take a more straight path through the DLD as they generally follow the fluid streamlines, whereas larger particles 320 are deflected to one side of the DLD by their interaction with carefully designed in-channel obstacles. The device can be used in conjunction with beads, where the beads which are the larger particles take a different path the cells alone. The cells can be incubated with the beads to generate bead-cell complexes and then the liquid (e.g., buffer, media, or another solution) can be applied to a DLD. The bead-cell complexes can be directed to a first path on the DLD and the unbound cells can be directed to a different path based on the size differential of the bead-cell complexes versus unbound cells. Dependent on the selection scheme (e.g., negative or positive selection), the bead-cell complexes or unbound cells can then be collected.

Size based selection can also be performed using inertial focusing. Inertial focusing can be performed in a microchannel in which a combination of forces, such as shear forces, fluid drag forces, or channel wall interaction forces, are applied to particles. These forces can be modulated by the curvature, the cross section, and/or other parameters of the channel. The size of particles allows for different strengths of forces to be applied to particles of different sizes. The resulting force differences cause smaller particles to be “focused” or generally moved to a location on the cross section of the channel, whereas larger particles are “focused” or generally moved to a different location on the cross section of the channel. By collecting particles from a certain area of the cross section of the channel, particles of a certain size can be collected. When inertial focusing is applied to beads, cells, and bead-cell complexes, the bead-cell complexes and unbound cells can be inertially focused and separated based on the size differential of bead-cell complexes and unbound cells.

Any combination of size-based separation, isolation, and/or selection methods can be used. For example, a selection process can include inertial focusing and a DLD device. In another example, a selection process can include first using a filter to isolate and/or select cells under a certain size followed by using inertial focusing.

Size selecting filters or membranes can have precise pore sizes suitable for selection of cells. For example, the pore sizes can be only large enough for a T-cell to pass through, but not any larger object. The pore size can be 10 μm, which is large enough for the T-cell to pass through but too small for a 20 μm bead to pass through. The pore size can be no more than 1 μm, 2 μm, 3 μm, 4 μm, 5 μm, 6 μm, 7 μm, 8 μm, 9 μm, 10 μm, 11 μm, 12 μm, 13 μm, 14 μm, 15 um, 16 μm, 17 μm, 18 μm, 19 μm, 20 μm, 21 μm, 22 μm, 23 μm, 24 μm, 25 μm, 30 μm, 35 μm, 40 μm, 45 μm, 50 μm, 55 μm, 60 μm, 65 μm, 70 μm, 75 μm, or 100 μm, or less. The pore size can be at least 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 um, 30 μm, 35 μm, 40 μm, 45 μm, 50 μm, 55 μm, 60 μm, 65 μm, 70 μm, 75 μm, or 100 μm, or more. The bead diameter can be larger than the pore, such the bead cannot pass through the pore. For example, the bead diameter can be at least 1 μm, 2 μm, 3 μm, 4 μm, 5 μm, 6 μm, 7 um, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 30 μm, 35 μm, 40 μm, 45 μm, 50 μm, 55 μm, 60 um, 65 μm, 70 μm, 75 μm, or 100 μm, or more.

As the filters are generally used to prevent objects from moving through, there can be a buildup of material on the filters or membranes that can clog the pores and may prevent cells from passing through the filter. This clogging can be alleviated by the use of cross-flow or other flow systems that include at least a partial flow vector that is orthogonal to the filter. The cells may be able to pass through the filters along a first axis and the liquid flow direction can include a component vector in a second axis that is orthogonal to the first axis. In this configuration, the flowing liquid (e.g., buffer, media, or another solution) can prevent any potentially clogging objects from being immobilized against the filter, and thus allow the pores to remain unblocked.

Buoyant solid supports (e.g., buoyant beads) can be used for cell isolation, separation and/or selection, and can be used in conjunction with filters, such as those described elsewhere in this disclosure. Buoyant solid supports can include hollow glass beads. These buoyant beads can float in a liquid medium for cell separation (e.g., a liquid of density/viscosity similar to water) and form a separate layer. As with other beads, a buoyant bead can comprise a binding agent that can bind to cells. As the buoyant beads generally float in the solution, the accompanying bound cells can be at least partially separated from the bulk solution. The unbound cells can then be washed away or otherwise removed such to generate a solution of, at least in part, bound beads.

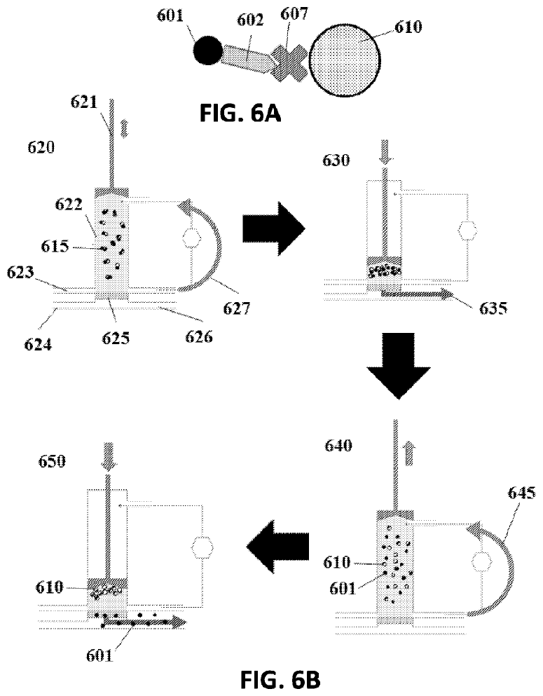

FIGS. 6A and 6B show an exemplary system 600 using buoyant beads for the separation, isolation, and/or selection of cells. The system 600 can include a plunger 621, a reaction chamber 622, an input channel 623, a wash channel 624, a filter 625, an output channel 626, and a recirculation channel 627. FIG. 6A shows a cell-bead complex 615 composed of a cell 601 and a buoyant bead 610 that includes a linking agent 607. The linking agent can be a streptavidin, or other molecule that includes a first part of a binding pair interaction. A binding agent 602 can be linked to the bead via the linking agent, and can be a biotinylated antibody or antibody fragment. A cell 601 can be bound to the bead via a binding agent 602 that has specificity to cell 601. FIG. 6B shows an exemplary method of using buoyant beads for cell separation, isolation, and/or selection. In configuration 620, the liquid (e.g., buffer, media, or another solution) in the reaction chamber 622 includes a plurality of beads 610 and cells 601. Buoyant beads 610 (e.g., hollow glass beads) that include a binding agent can be added to the reaction chamber manually, e.g., via a syringe, automatically, e.g., via continuous or incremental flow, or by other methods. The plunger 621 can be used to modulate the active volume of the reaction chamber 622 and apply pressure on the liquid causing liquid (e.g., buffer, media, or another solution) to flow out of the reaction chamber 622. In configuration 620, the plunger 621 is not activated. The filter 625 can be located at the bottom of the reaction chamber 622, between the reaction chamber 622 and the wash and output channels 624 and 626.

The input channel 623 and the output channels 624, 626 can run in parallel and be in fluidic communication with the filter 625 disposed between them to allow liquid (e.g., buffer, media, or another solution) to move freely between the two channels, but prevent the movement of larger objects, such as bead/cell complexes between the channels. The cells can be circulated into the reaction chamber 622 through the recirculation channel 627. The buoyant beads 610 can bind cells 601 forming bead-cell complexes 615. The pores of the filter 625 can be smaller than a buoyant bead 610 and/or a bead-cell complex 615, and can allow unbound cells to pass through the filter 625 without the buoyant beads 610 and/or bead-cell complex 615 passing through.

As shown in configuration 630, the plunger 621 can be activated to push at least a portion of the liquid (e.g., buffer, media, or another solution) in the reaction chamber 622 through the filter 625 while the buoyant beads 610 and bead-cell complexes 615 remain above the filter 625. The buoyant properties of the beads 610 may reduce or prevent clogging of the filter 625. Unbound cells that pass through the filter 625 can exit through the output channel as denoted by 635. The plunger 621 can be returned to its starting position setting the active volume of the reaction chamber 622 to its initial state, as shown in configuration 640. New liquid (e.g., buffer, media, or another solution) can be added to the reaction chamber 622 through the input channel 623 (e.g., when the plunger is pulled and draws liquid (e.g., buffer, media, or another solution) into the reaction chamber) and bioreactor system content can be recirculated via the recirculation channel 627. The new liquid can include additional cells to bind and/or wash solution to remove contaminants, and the process shown in configurations 620, 630, and 640 can be repeated. Based at least on pH level, ionic strength or other chemical characteristics, the wash solution used can affect the purity and yield of isolated and/or selected cells. For example, a higher ionic concentration may disrupt non-specific interactions and result in a more stringent wash condition. Similarly, pH levels that are higher or lower than a neutral pH can alter interactions and allow for a more stringent wash condition. Additionally, the speed at which the wash solution is applied, as well as the agitation of the wash solution, can modulate the purity and yield. Stringent wash conditions can reduce overall yield and can improve purity, whereas less stringent wash conditions can result in a higher overall yield and reduce purity. The process can be repeated at certain frequencies and/or intervals, which can be implemented manually or automatically. For example, the frequencies and/or intervals can be defined and/or (automatically) adjusted based on the cell concentration and/or concentration of cell/bead complexes in the reaction chamber 622. The repeating cycles can include a plunger push to filter a portion of the bioreactor volume, followed by a plunger pull to draw in a smaller volume of wash buffer, followed by a plunger push (e.g., 2 steps forward, half step back), to defoul the filter, for example.

The liquid can include elution buffer that disrupts the interaction between the binding agent and the cells, or the interaction between the binding agent and the bead. For example, when the cells and beads are bound by biotin-streptavidin interaction, the liquid (e.g., buffer, media, or another solution) can include free biotin to elute the cells and/or beads. The free biotin can have a higher binding affinity to streptavidin than the biotin bound to the streptavidin, and can displace the bound biotin. Configuration 640 shows the reaction chamber 622 with the resulting solution after addition of an elution buffer to disrupt the interactions between the cells and beads of the bead-cell complexes 615 thereby releasing cells 601 from beads 610. As shown in configuration 650, after elution of cells 601, the plunger 621 can be activated, thereby modulating the active volume of the reaction chamber 622 and pushing at least a portion of the eluted cells 601 through the filter 625. The buoyant properties of the beads 610 may reduce or prevent clogging of the filter 625, and the size of the beads 610 may prevent from passing through the filter 625 to avoid interference with downstream processes or products, for example.

The elution buffer can include a reagent that disrupts the interactions of the bead and the cells. As shown in FIG. 6A, the interaction can include multiple molecules, for example, linking agent 607, and binding agent 602. The reagent can disrupt the interaction between the linking agent 607 and the bead 610, the linking agent 607 and the binding agent 602, the binding agent 602 and the cell 601, or any combination thereof. As described above, a linking agent 607 can be a streptavidin and the binding agent 602 can include a biotinylated molecule. In some implementations, the addition of free biotin can disrupt this interaction, as the free biotin can outcompete the biotinylated molecule and displace the biotinylated molecule from its binding with streptavidin. The elution buffer can include salts that disrupt the interactions. The elution buffer can include bases or acids, or include a different pH such that the interactions are disrupted. For example, an elution buffer can include a lower pH than the binding buffer. For example, an elution buffer can include a higher pH than the binding buffer.

The methods disclosed herein can use dissolvable solid supports (e.g., dissolvable beads) for the isolation, separation, or selection of cells. The solid supports may be able to bind cells forming solid support/cell complexes that can be separated or isolated from unbound cells. The dissolvable properties of the solid supports allow for the solid supports to be removed by a reaction (e.g., chemical or biological), which may provide an advantage over non-dissolvable solid supports. For example, the presence of solid supports may be undesirable in downstream product or may interfere with downstream processes. The dissolution of the solid support can also provide for a method to disrupt an interaction formed between the solid support and a cell. Other methods or processes to separate cells from solid supports, after they have been bound together, may introduce conditions that could impact cell viability.

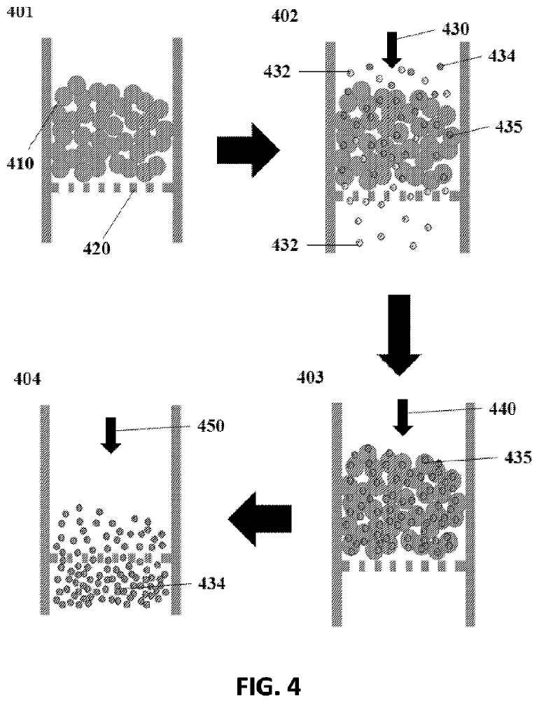

An exemplary method for the isolation, separation, and/or selection of cells using dissolvable beads is shown in FIG. 4. The dissolvable beads 410 can be composed of any dissolvable material, such as dissolvable polymer, and include a binding agent that can bind to a cell of interest. The dissolvable beads 410 can be packed into a column as shown in configuration 401. The column of dissolvable beads 410 can be packed at a density to allow for a liquid (e.g., buffer, media, or another solution) to flow through the column and pass by the dissolvable beads 410.

A sample 430 including cells 432 and cells 434, for example, can be added to the column as shown in configuration 402. The cells 432 and 434 contact at least some of the dissolvable beads 410 as the sample flows through the column. The dissolvable beads 410 can include binding agents that are configured to bind to cells 434 but do not bind to cells 432. As the cells 434 flow through the column and contact dissolvable beads 410, at least some of the cells 434 may be bound to dissolvable beads 410 via the binding agent, forming bead-cell complexes 435. The size of dissolvable bead and density or packing parameters of the column can be adjusted such that cells can pass though the column while making significant contact with the dissolvable beads, allowing for a large portion of cells to bind to the dissolvable beads. Similarly, size of dissolvable bead and density or packing parameters of the column can allow cells that are not of interest to flow through the column such that the column volume includes primarily cells of interest.

At the bottom of the column, a membrane or filter 420 can be used to allow for unbound cells to pass through, while preventing the dissolvable beads 410 or bead-cell complexes 435 to pass through. Cells that do not bind to dissolvable beads can pass through the filter 420 and exit the column, such as cells 432, while cells 434 may be bound to dissolvable beads 410 forming bead-cell complexes 435 as discussed above. Once cells 434 have bound to dissolvable beads 410, the column can be washed with a wash solution 440, for example, to remove any contaminants or residual material that may be present in the column, while cell-bead complexes 435 remain in the column, as shown in configuration 403. Based at least on pH level, ionic strength or other chemical characteristics, the wash solution used can affect the purity and yield of isolated and/or selected cells. For example, a higher ionic concentration may disrupt non-specific interactions and result in a more stringent wash condition. Similarly, PH levels that are higher or lower than a neutral pH can alter interactions and allow for a more stringent wash condition. Additionally, the speed at which the wash solution is applied, as well as the agitation of the wash solution, can modulate the purity and yield. Stringent wash conditions can reduce overall yield and can improve purity, whereas less stringent wash conditions can result in a higher overall yield and reduce purity.

To collect cells 434, the cells 434 can be eluted out of the column, for example. This can be performed by dissolving the dissolvable beads 410 of the bead-cell complexes 435 thereby releasing cells 434 to flow out of the column, allowing for cells 434 to be harvested or otherwise directed to further processing step(s). The dissolution of dissolvable beads 410 can be initiated by applying a reagent 450 configured to dissolve the bead as shown in configuration 404. For example, a dissolvable bead can be an alginate bead and citrate can be used to dissolve the bead.

Elution of cells by dissolving beads from bead-cell complexes can be advantageous compared to other methods of elution. The connection between the cells and beads using antibodies can result in a strong interaction of the antibody and cell that can be difficult to disrupt. For example, disruption of the connection can include the use of reagents that are unfavorable to cell viability, potentially resulting in lower quality of the released cells. The use of dissolvable beads can offer advantages over other systems that rely on the disruption of binding agent to cell interaction. By dissolving the beads, disruption of a cell-antibody interaction is not needed to de-bead the cells. Additionally, the beads may provide additional structure that allow for the binding agents (e.g., Fabs) to be stable and the dissolving of the bead can destabilize the binding agent and allow for release of the cell.

The above description relates to a positive separation, isolation, and/or selection of cells; however the systems and methods can also be used for negative selection. For example, dissolvable beads in a column can be configured not to bind cells of interest, but other cells within a liquid (e.g., buffer, media, or another solution) flowing through the column. Here, non-targeted cells can bind to the dissolvable beads while cells of interest may pass through the column. The bead-cell complexes can then be dissolved and the released non-targeted cells can be washed out of the column. The column space can then be used for a new cell isolation, separation, and/or selection process, thereby allowing for efficient recycling of the column space.

The described methods can also use dissolvable magnetic solid supports (e.g., dissolvable magnetic beads), which can provide similar advantages as dissolvable beads, and include magnetic characteristics. Dissolvable magnetic solid supports can be manipulated and isolated using a magnet, which can be leveraged to isolate or select cells. Dissolvable magnetic solid supports can also include binding agents, such as antibodies. A binding agent can bind cells to dissolvable magnetic solid supports and a magnet can then be used to manipulate the dissolvable magnetic solid supports and cells bound thereto. For example, a magnet can be used to collect or pull out the dissolvable magnetic solid supports from a liquid, e.g., the magnet can be used to attract the dissolvable magnetic solid supports and isolate the dissolvable magnetic beads, and any cells bound to them, from a liquid (e.g., buffer, media, or another solution). The isolation can be performed using bulk separation or flow separation methods. For example, a solution of dissolvable magnetic solid supports can be added to a liquid (e.g., buffer, media, or another solution) in a vessel (e.g., a tube, container, etc.) to interact with other objects in the liquid. A magnet (external or internal) can be placed such that the dissolvable magnetic solid supports can become attracted to the magnet thereby pulling the dissolvable magnetic solid supports to a certain location. In another example, dissolvable magnetic solid supports can be added to a liquid (e.g., buffer, media, or another solution) flowing through a channel (e.g., a microchannel) to interact with other objects in the liquid. A magnet (external or internal) can be placed at a certain segment of the channel attracting the dissolvable magnetic solid supports and immobilizing the dissolvable magnetic solid supports while other objects continue to flow past the magnet.

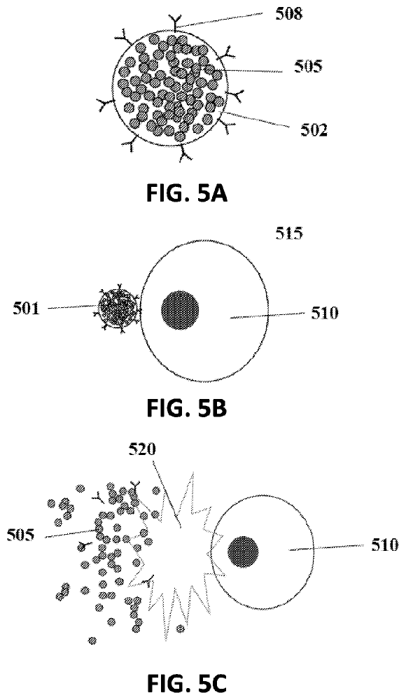

FIGS. 5A-5C illustrate exemplary dissolvable magnetic beads and their uses. FIG. 5A shows an exemplary dissolvable magnetic bead 501 that is composed of a dissolvable polymer 502, such as alginate, magnetic particles 505, and a binding agent 508, such as an antibody. The dissolvable magnetic beads 501 can be mixed with cells. The dissolvable beads 501 can bind certain cells, for example as discussed with respect to FIGS. 1, 4, and 6A. FIG. 5B shows a dissolvable magnetic bead 501 bound to a cell 510 forming a bead-cell complex 515. The bead-cell complexes 515 (and unbound dissolvable magnetic beads 501) can be isolated, separated, and/or selected using a magnetic force. The magnetic force can be generated by a magnet (external or internal) that is positioned such that the bead-cell complexes 515 (and dissolvable magnetic beads 501) are attracted by the magnet. Upon separation, isolation, and/or selection of the bead-cell complexes 515, a wash solution can be added to remove contaminants. Based at least on pH level, ionic strength or other chemical characteristics, the wash solution used can affect the purity and yield of isolated and/or selected cells. For example, a higher ionic concentration may disrupt non-specific interactions and result in a more stringent wash condition. Similarly, pH levels that are higher or lower than a neutral pH can alter interactions and allow for a more stringent wash condition. Additionally, the speed at which the wash solution is applied, as well as the agitation of the wash solution, can modulate the purity and yield. Stringent wash conditions can reduce overall yield and can improve purity, whereas less stringent wash conditions can result in a higher overall yield and reduce purity.

As shown in FIG. 5C dissolvable magnetic beads 501 in the bead-cell complexes 515 can be dissolved thereby releasing the cells 510. For example, a dissolving reaction can be performed by adding a reagent 520 that can dissolve the dissolvable magnetic beads 501 thereby releasing the cells 510 (e.g., for collection) and the magnetic particles 505. The resulting liquid (e.g., buffer, media, or another solution) including the released cells 510 and magnetic particles 505 can be subjected to a magnetic force (during or after dissolution) to capture the magnetic particles. These captured magnetic particles can then be discarded or recycled to generate new magnetic dissolvable beads.

Magnetic bead 501 can be a double layer bead. An inner layer can have magnetic particles 505 that are embedded, adsorbed, or otherwise attached to the inner layer. The inner layer can be non-dissolvable or resistant to dissolution. An outer layer can be dissolvable and include binding agents 508. When the bead is subjected to dissolution, as show in FIG. 5C, the outer layer can dissolve and release the cell, and the inner layer can remain intact allowing the magnetic particle to remain together. The inner layer can then be captured to be discarded or recycled to generate new magnetic dissolvable beads.

Another method of separation, isolation and/or selection of cells can utilize matrices (e.g., polymer matrices) as opposed to beads. Polymer matrices can include binding agents. Polymer matrices can be generated by introducing unpolymerized polymer precursors into a channel, for example. The unpolymerized polymer precursors can by polymerized by the addition of a polymerization agent. A binding agent can be integrated into the polymer matrix. A liquid (e.g., buffer, media, or another solution) of unpolymerized polymer precursors and a binding agent can be polymerized to generate a polymer matrix comprising a binding agent. The polymer matrix is porous (e.g., an “open pore structure” similar to a filter) allowing, for example, for cells to flow though. For example, similarly to the method shown in FIG. 4, cells can flow into a reaction area, in this case polymer matrices, as opposed to the beads shown in FIG. 4. The cells can then interact with the polymer matrices and become bound to the matrices. Cell that do not bind to the polymer matrices can flow through. The polymer matrix can be dissolved to release the bound cells.

The methods of using a polymer matrix can be performed in a microchannel. For example, a liquid (e.g., buffer, media, or another solution) of unpolymerized polymer precursors and binding agents can be introduced into a microchannel. A polymerization agent can then be added to initiate polymerization and generate the polymer matrix. Cells flow through the microchannel to interact with the polymer matrix and cells can bind with the binding agent. Cells that do not interact with the binding agent can flow through the polymer matrix. The polymer matrix can be washed to remove contaminants. The polymer matrix can then be dissolved to release cells from the polymer matrix and, for example, allow the cells to be collected or moved through microchannels into another module or system for further processing.

Washing the matrix can be performed using different wash solutions to modulate purity and yield of the resulting isolated/selected cells. Based at least on pH level, ionic strength or other chemical characteristics, the wash solution used can affect the purity and yield of isolated and/or selected cells. For example, a higher ionic concentration may disrupt non-specific interactions and result in a more stringent wash condition. Similarly, PH levels that are higher or lower than a neutral pH can alter interactions and allow for a more stringent wash condition. Additionally, the speed at which the wash solution is applied, as well as the agitation of the wash solution, can modulate the purity and yield. Stringent wash conditions can reduce overall yield and can improve purity, whereas less stringent wash conditions can result in a higher overall yield and reduce purity.

The methods can be iterated such to improve isolation, separation, and/or selection efficiency and/or allow for multiple phenotypes (or cell types) to be captured or isolated. For example, after performing the methods to capture cells and dissolve a polymer matrix to release the cells, one or more additional iterations of the isolation, separation, and/or selection processes can be performed. The binding agent in the additional polymer matrix(es) can be the same or different from the binding agent in the previous polymer matrix. For example, a first polymer matrix can comprise an antibody for binding a T-cell, and the second polymer matrix can comprise the same antibody. This can improve the overall purity of the resulting cells. For example, after a first iteration of the method, a small amount of cells that did not interact with the polymer matrix or binding agent (or were bound by non-specific interaction) may remain, and be retained in the matrix after washing. These cells may be released along with the cells of interest that were specifically bound with the binding agent. Additional iterations can filter out these cells (or non-specifically bound cells).

In another example, a first polymer matrix can include an antibody for binding a T-cell, and the second polymer matrix can include a different antibody targeting a subset of the initially isolated T-cells. The two antibodies can have different epitopes such that the resulting selected T-cell comprises includes a phenotype that has polypeptides corresponding to the different epitopes. This can provide for improved overall purity of the resulting cells. The different binding agents can also allow for improved isolation, separation, and/or selection of specific subtypes of cells. For example, a first binding agent in the first matrix can be configured to bind a protein that is present in all T-cell, whereas a second binding agent in the second matrix can be configured to bind a protein specifically expressed on activated T-cells. In another example, a first binding agent in the first matrix can be configured to bind a protein that is present in all T-cells, whereas a second binding agent in the second matrix can be configured to bind the same protein (e.g., by binding a same epitope or different epitope of the same protein). The first matrix and second matrix can include the same exact antibody, or different antibodies to the same protein. This repeated process may allow for improved isolation, separation, and/or selection by capturing additional cells that did not bind initially to the first matrix.

Iterative processes can be performed in a channel (e.g., a microchannel) or multiple channels (e.g., microchannels) that form a microfluidic system. Cells can flow in the channel and/or junctions of the channels allowing for the addition of polymer precursors, binding agents. polymerization agents and/or de-polymerization agents, for example to generate polymer matrices in the channel(s). Multiple junctions can be used to iterate the process, with the addition of matrices being formed on demand in the channel(s), and cells being collected (or discarded) based on the flow and design of the system.

Cells can be subject to multiple rounds of separation, selection and/or isolation and the methods described in this disclosure can be combined to be performed sequentially (in any order) and/or in parallel. For example, cells can be first subjected to a size-based selection process and then to another selection process based on binding the cells to a particular antibody. As another example, cells can be first subjected a selection process based on the presence of a first phenotypic feature and then to another selection process based on the presence of a second phenotypic feature. In another example, beads used for size-based methods can be dissolvable beads, magnetic beads, magnetic dissolvable beads, buoyant beads, or beads comprising other characteristics. The beads can thus be subjected to magnetic fields, dissolution reactions, and/or other reactions as described for other methods herein. The use of multiple different methods for the separation, isolation and/or selection of cells may improve efficiencies, efficacy, and quality of the process(es). For example, dissolvable beads that bind to cells of interest can be subjected to size-based separation, selection and/or isolation methods. The cells of interest can bind to the dissolvable beads to form bead-cell complexes, which then can be subjected to DLD to separate the bead-cell complexes from unbound cells. To collect the cells of interest (which are bound to the dissolvable beads), the dissolvable beads of the bead-cell complexes can be dissolved to release the cells of interest. As some size-based selection methods may be unable to distinguish between beads alone and bead-cell complexes, dissolution of the beads can allow for the beads (whether bound or not) to be dissolved after size selection process(es) and only the cells remain.

In various aspects, the methods and systems comprise the use of solid supports that are able to bind or capture cells. For example, a solid support can be a bead, a polymer matrix, or other material. The solid support can include, for example, glass, plastic, collagen, agarose, alginate, PLGA, polyacrylamide, or other polymer. The solid support (e.g., a dissolvable bead) can include a dissolvable polymer. The dissolvable solid support can be dissolved and in some instances dissociate components of the dissolvable support thereby releasing the components into a liquid (e.g., buffer, media, or another solution). The dissolvable solid support can be constructed of polymeric materials that can be dissolved using a chemical stimulus. For example, the dissolvable solid support can be an alginate bead. Alginate is a natural polymer that can form a gel when dissolved in water. The addition of certain salts (such as those containing Ca²⁺, or other multivalent cations) can initiate the gelation process to form beads, which may encapsulate particles. For example, alginate can be used to encapsulate magnetic particles to form a dissolvable magnetic bead. Alginate can be dissolved and release any bound or encapsulated objects. For the dissolution process citrate, EDTA, or other chelator can be used to reverse the gelation process thereby dissolving the bead. In some cases, alginate can be dissolved using alginase. Reagents can be added to a liquid (e.g., buffer, media, or another solution) including the dissolvable beads to initiate the dissolution process, resulting in the release of any bead-bound objects.

In various aspects, a binding agent can be used to bind to cells. A binding agent can be a protein or a polypeptide that comprises an affinity to another molecule or macromolecule. A binding agent can be an antibody, or fragment thereof. For example, a binding agent can be a scFv (single chain variable fragment), nanobody, or Fab (fragment antibody) domain. A binding agent can be specific to a particular protein such that a cell type (or phenotype) can be separated, selected and/or isolated. For example, a binding agent can include affinity for a T-cell. A binding agent can bind to CD3, or other proteins expressed by a cell, such as a T-cell. For example, the binding agent can bind to, for example, CD3, CD4, CD8, CD14, CD19, CD20, CD22, CD25, CD27, CD28, CD34, CD45, CD45RA, CD45RO, CD56, CD62L, CD127, CCR7, TCR, TCR alpha/beta, TCR gamma/delta, or the polypeptide or molecule expressed by a cell or otherwise present on the surface of a cell. The cells can have a particular phenotype, for example, CD28+, CCR7+, CD27+, CD127+ cells. Cells can interact with the binding agent, and the T-cells in the cells can bind to the binding agent. T-cells can then be separated, isolated, and/or selected by various reactions as those described elsewhere herein such as size-based, washing, dissolution, and/or magnetic cell separation, isolation, and/or selection processes.

A binding agent can be connected to a solid support via a linker. A binding agent can be coupled to a solid support using non-covalent or covalent interactions to generate a linker. For example, a binding agent can be directly coupled with a covalent bond linking the binding agent directly to a solid support. The covalent bond can be generated using “click” chemistry or other reactions known in the art to generate a covalent bond. In another example, a binding agent can be coupled to a solid support via non-covalent interactions. For example, a binding agent and solid support can be coupled using a streptavidin (or other avidin) and biotin. The binding agent can include a biotin and the solid support can be a streptavidin bead. The non-covalent interaction can be disrupted to release the binding agent from the solid support. For example, in the case of a biotin-streptavidin interaction, free biotin can be added to the liquid (e.g., buffer, media, or another solution) to disrupt the interaction of the biotinylated binding agent and the streptavidin bead. The free biotin can outcompete the biotinylated binding agent to release the biotinylated binding agent from the solid support. The biotinylated binding agent can include a modified biotin that has a weaker binding affinity to streptavidin. Such non-modified biotin can more efficiently outcompete the modified biotin for streptavidin. For example, the modified biotin can be a desthiobiotin. A modified biotin can also have a stronger binding affinity to streptavidin than a non-modified biotin. The modified biotin with a stronger binding affinity to streptavidin can more efficiently outcompete the non-modified biotin for streptavidin. As described throughout the disclosure, the binding agent can be bound to a cell, and the release of the binding agent from the solid support can allow the cell to be released from the solid support. The binding agent can be initially linked to the solid support with a non-covalent interaction and the release of the binding agent can be performed by introduction of a molecule that can compete for the binding of the solid support. For example, the linker can be composed of a streptavidin bound to a modified biotin with a weaker affinity to streptavidin as compared to non-modified biotin. The introduction of free non-modified biotin can compete with the modified biotin to displace the modified biotin and disrupt the modified biotin binding interactions with the streptavidin. In another example, the linker can be composed of a streptavidin bound to a non-modified biotin. The introduction of free modified biotin that has a stronger affinity to streptavidin as compared to non-modified biotin can compete with the non-modified biotin to displace the non-modified biotin and disrupt the non-modified biotin binding interactions with the streptavidin.

The non-covalent interaction can include an antibody-antigen interaction. A support can comprise an antigen and the binding agent can comprise an antibody. For example, a linker can include a biotin and an anti-biotin antibody. The linker can be a streptavidin and an anti-streptavidin antibody. The linker can comprise a dissolvable matrix (e.g., a dissolvable polymer matrix). A dissolving reagent (e.g., reducing agent) can be used to dissolve a polymer matrix shell. In some cases, a dissolving reagent (e.g., reducing agent) can comprise tris(2-carboxyethyl) phosphine (TCEP), beta-mercaptoethanol (BME), and/or dithiothreitol (DTT).

A solid support can be functionalized with linking agents or moieties that can couple to other molecules, such as the binding agent. A solid support can include functionalization such as the attachment or adsorption of streptavidin, biotin, protein A, protein G, maltose binding protein (MBP), or other proteins that are able to bind another molecule. A solid support can include reactive groups to allow for functionalization or attachment to other molecules. For example, a solid support can include an azide, amino, alkynyl, alkenyl, or other reactive moiety.

Polymer precursors can be conjugated to moieties that allow for interaction with binding agents. Polymer precursors can, for example, include reactive moieties, such as azides, alkynes, alkenes, or other reactive moieties known in the art. Binding agents can include reactive moieties that can react with reactive moieties on polymer precursors. As such, the reactive moieties can react to link the binding agent with the polymer precursors (or polymerized polymer). The placement of reactive moieties on binding agents can allow for a binding agent to properly display the binding end away for the matrix. For example, an antibody can be used as a binding agent and the Fc domain can comprise a reactive moiety. The Fc domain can react and become linked with the polymer matrix such that the antibodies variable regions are available (e.g., not sterically blocked or hindered) to bind to cells passing through the matrix. Similarly, non-covalent binding pairs can be present on the polymer precursors and binding agents to link the polymer and the binding agents, and can be placed to orient the binding agents in a desired orientation. For example, a polymer can comprise biotin moieties and an antibody can be conjugated to a streptavidin. Alternatively, a polymer can include streptavidin and an antibody can be conjugated to a biotin, for example.

The polymer precursor can also act as a structural component for the binding agent. For example, a protein (e.g. Fab) that independently has low avidity to the antigen can have improved binding affinity when co-polymerized with the polymer matrix. Upon dissolution of a matrix, the binding affinity may be reduced, thereby disrupting the binding agent-cell interaction and releasing the cell. For example, a binding agent can comprise an antibody variable region domain without a corresponding Fc domain (e.g. Fab). The polymerized matrix can provide structural stability similar to a Fc domain and the antibody variable region domain can bind to cells. Upon dissolution of the matrix, the antibody variable region domain can lose structural stability resulting in the loss of binding affinity or binding avidity and cause a release of the cells.

A solid support can include magnetic particles or other magnetic materials. A solid support including magnetic particles can be manipulated using magnetic fields (e.g., magnets). The magnetic particles can be magnetic or paramagnetic nanoparticles. Magnetic particles can be embedded in a solid support. Magnetic particles may be coupled to a solid support via a linker. Magnetic particles may be adsorbed to the surface of the solid support.

Magnetic dissolvable beads can be generated by using a microfluidic droplet generator that co-injects a liquid solution including polymer precursors and oil through an orifice into a channel (e.g., a microchannel). Magnetic nanoparticles can be mixed in with the liquid solution. Polymer precursors can crosslink and form a suspension of dissolvable gel beads filled with magnetic particles suspended in an oil phase. Magnetic beads can then be removed from the oil phase and resuspended in a solution for used for downstream processes. For example, a liquid solution of alginate and magnetic particles suspending in mineral oil can be used in a microfluidic droplet generator to form magnetic alginate beads.

Magnetic beads can also be generated using spray drying. Spray drying of polymer precursor droplets co-mixed with magnetic nanoparticles can be performed to generate magnetic beads. After drying, these beads can be re-constituted in a buffer (e.g., phosphate-buffered saline or any other buffer).

Target cells may be incubated and/or labeled with lipid microbubbles and separated through gravity settling for at least about, at most about, or about 1 minute, 2 minutes, 3 minutes, 4 minutes, 5 minutes, 6 minutes, 7 minutes, 8 minutes, 9 minutes, 10 minutes, 12 minutes, 15 minutes, 18 minutes, 20 minutes, 22 minutes, 25 minutes, 30 minutes, 45 minutes, 60 minutes, or a range between any of these values. Flushing of non-target cells and repeated separations can increase purity of the target T cells. A pressure source can be used to remove a separation particle and/or linker between the separation particle and binding agent. In some cases, a pressure pulse used to collapse or dissolve the lipid microbubble can be at least about 0.1 bar, at least about 0.2 bar, at least about 0.3 bar, at least about 0.4 bar, at least about 0.5 bar, at least about 1 bar, at least about 1.1 bar, at least about 1.2 bar, at least about 1.3 bar, at least about 1.4 bar, at least about 1.5 bar, at least about 1.6 bar, at least about 1.7 bar, at least about 1.8 bar, at least about 1.9 bar, at least about 2 bar, at least about 2. 1 bar, at least about 2.2 bar, at least about 2.3 bar, at least about 2.4 bar, at least about 2.5 bar, at least about 3 bar, at least about 4 bar, at least about 5 bar, or greater than 5 bar of pressure. In some cases, a pressure pulse used to collapse or dissolve the lipid microbubble can be at most about 5 bar, at most about 4 bar, at most about 3 bar, at most about 2.5 bar, at most about 2.4 bar, at most about 2.3 bar, at most about 2.2 bar, at most about 2.1 bar, at most about 2 bar, at most about 1.9 bar, at most about 1.8 bar, at most about 1.7 bar, at most about 1.6 bar, at most about 1.5 bar, at most about 1.4 bar, at most about 1.3 bar, at most about 1.2 bar, at most about 1.1 bar, at most about 1 bar, at most about 0.5 bar, at most about 0.4 bar, at most about 0.3 bar, at most about 0.2 bar, at most about 0.1 bar, or less than about 0.1 bar of pressure. In some cases, a pressure pulse used to collapse or dissolve the lipid microbubble can be from about 0.1 bar to about 5 bar of pressure. In some cases, a pressure pulse used to collapse or dissolve the lipid microbubble can be from about 0.1 bar to about 0.2 bar, about 0.1 bar to about 0.3 bar, about 0.1 bar to about 0.4 bar, about 0. 1 bar to about 0.5 bar, about 0.1 bar to about 1 bar, about 0.1 bar to about 1.5 bar, about 0.1 bar to about 2 bar, about 0.1 bar to about 2.5 bar, about 0.1 bar to about 3 bar, about 0.1 bar to about 4 bar, about 0.1 bar to about 5 bar, about 0.2 bar to about 0.3 bar, about 0.2 bar to about 0.4 bar, about 0.2 bar to about 0.5 bar, about 0.2 bar to about 1 bar, about 0.2 bar to about 1.5 bar, about 0.2 bar to about 2 bar, about 0.2 bar to about 2.5 bar, about 0.2 bar to about 3 bar, about 0.2 bar to about 4 bar, about 0.2 bar to about 5 bar, about 0.3 bar to about 0.4 bar, about 0.3 bar to about 0.5 bar, about 0.3 bar to about 1 bar, about 0.3 bar to about 1.5 bar, about 0.3 bar to about 2 bar, about 0.3 bar to about 2.5 bar, about 0.3 bar to about 3 bar, about 0.3 bar to about 4 bar, about 0.3 bar to about 5 bar, about 0.4 bar to about 0.5 bar, about 0.4 bar to about 1 bar, about 0.4 bar to about 1.5 bar, about 0.4 bar to about 2 bar, about 0.4 bar to about 2.5 bar, about 0.4 bar to about 3 bar, about 0.4 bar to about 4 bar, about 0.4 bar to about 5 bar, about 0.5 bar to about 1 bar, about 0.5 bar to about 1.5 bar, about 0.5 bar to about 2 bar, about 0.5 bar to about 2.5 bar, about 0.5 bar to about 3 bar, about 0.5 bar to about 4 bar, about 0.5 bar to about 5 bar, about 1 bar to about 1.5 bar, about 1 bar to about 2 bar, about 1 bar to about 2.5 bar, about 1 bar to about 3 bar, about 1 bar to about 4 bar, about 1 bar to about 5 bar, about 1.5 bar to about 2 bar, about 1.5 bar to about 2.5 bar, about 1.5 bar to about 3 bar, about 1.5 bar to about 4 bar, about 1.5 bar to about 5 bar, about 2 bar to about 2.5 bar, about 2 bar to about 3 bar, about 2 bar to about 4 bar, about 2 bar to about 5 bar, about 2.5 bar to about 3 bar, about 2.5 bar to about 4 bar, about 2.5 bar to about 5 bar, about 3 bar to about 4 bar, about 3 bar to about 5 bar, or about 4 bar to about 5 bar of pressure.

In various aspects, cells are derived from a subject (e.g., collected as a sample from a subject). The subject can be a human. The subject can be an individual that is suffering from a disease, disorder, or other condition. For example, the subject can have cancer or an autoimmune disorder. The sample can be a blood sample or bone marrow sample, for example. The sample can be subjected to processes to isolate and/or select for a type of cells, for example, a leukocyte or erythrocyte. The sample can comprise immune cells. For example, the sample can include lymphocyte, T-cells, NK cells, B cells, leukocytes, macrophages, dendritic cells, monocytes, mast cells neutrophils or other immune cells. The sample can comprise naïve T-cell or B-cells. The sample can include mature T-cell or B-cells. The sample can include stem cells.

In various aspects, systems are used to perform the methods described herein. The systems can comprise components for flowing cells and reagents, as well as chambers and columns for allowing cells to bind to solid supports. For example, a system can comprise a chamber for holding beads and cells, an input channel fluidically connected to the chamber, an output channel fluidically connected to the chamber; and a filter disposed at an entrance of the output channel from the chamber. A plunger can be integrated in the chamber, which can be used to apply pressure to fluid in the chamber to move fluid to the output channel. A system can include a reaction chamber, a syringe, and a syringe plunger. A reaction chamber can be connected to a wash input channel. A reaction chamber can be connected to a channel allowing for recirculation of fluid, or exchange and/or addition of fresh fluid into a reaction chamber.

Various systems can use microfluidic channels as input and output channels. Microfluidic channels can allow for the movement of fluids in small volumes. Microchannels can be advantageous in generating complex fluidic connections in a small space that can otherwise be inefficient to generate or manipulate on a macro scale.

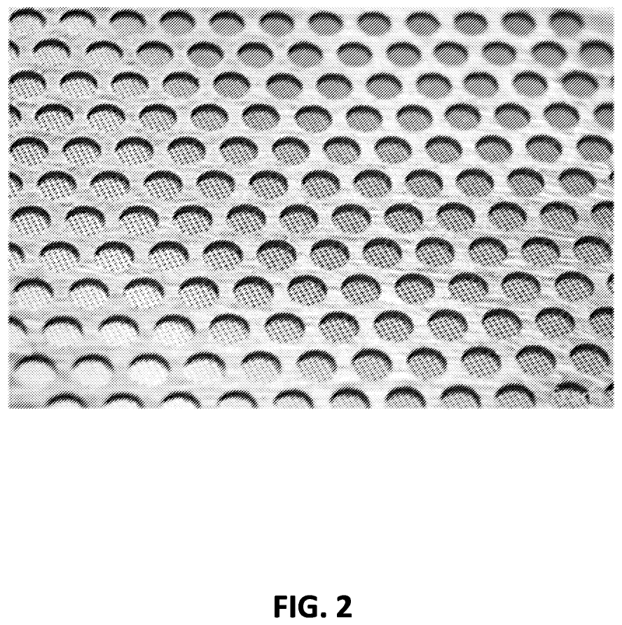

Various systems can use filters or membranes, such as those described herein. A filter can include a sieve. A filter can comprise a plurality of pores of a specific size. A filter can be used to prevent large objects from entering or exiting a reaction chamber. For example, large contaminants can be prevented from entering a reaction chamber. A filter can be used to prevent large objects from entering or exiting a column. For example, a filter can prevent solid supports from leaving a column so that the column remains packed. Filters or membranes can be made from fibers or metals. Metals can be machined to have precise pore sizes. FIG. 2 shows an exemplary microfilter created by etching stainless steel. Additionally, other techniques can be used such as silicon machining, photoetching, or other methods known in the art to generate precise structures in material, such as the generation of holes of a particular size.

Methods and systems for separating, isolating and/or selecting cells can provide cells of interest for research and development of cell-based diagnostics and therapeutics

To generate cell-based therapies, cells that can be used as precursors to therapeutic cells can be initially isolated or selected from a mixture of cells. Samples derived from a subject (e.g., a whole blood sample), can include different types of cells, of which only some may be useful for generating a cell that can confer a therapeutic benefit. As such, the ability to effectively separate, isolate, and/or select a cell of interest is needed.

Cell separation, isolation, and/or selection can also be used for diagnostic purposes such as identification of cell markers or specific phenotypes in a subject. For example, a subject can be suspected of having a disease and isolation of a specific cell type can be used to de-convolute or simplify diagnosis or detection of biomarkers present in one or more cell types.

Additionally, cell separation, isolation and/or selection can be beneficial for generating substantially pure cultures of cells for assays. For example, primary cells from a subject can be isolated and various molecules or stimuli can be applied to the cells to determine the response of the cells to the molecules or stimuli.

The systems, methods, compositions, can be flexible and can accommodate a diversity of different cell types based at least on the selection parameters and structures described herein. For example, the system can be able to accommodate a diversity of different immune cell types (T cells, NK cells, B cells), and can be directed at different protein targets (CD19, BCMA, etc.) that are expressed by the cells.

The methods described herein may separate target T cells from other white blood cells of a sample and result in greater purity than conventional methods or negative controls (e.g., no binding agent). In some cases, the methods described herein result in a purity of CD4+ T cells of at least about 50%, at least about 55%, at least about 60%, at least about 65%, at least about 70%, at least 75%, at least about 80%, at least about 85%, at least about 90%, at least about 95%, at least about 96%, at least about 97%, at least about 98%, at least about 99%, or greater than 99%. In some cases, the methods described herein result in a purity of CD4+ T cells of at most about 99%, at most about 98%, at most about 97%, at most about 96%, at most about 95%, at most about 90%, at most about 85%, at most about 80%, at most about 75%, at most about 70%, at most about 65%, at most about 60%, at most about 55%, at most about 50%, or less than about 50%. In some cases, the methods described herein result in a purity of CD4+ T cells from about 50% to about 100%. In some cases, the methods described herein result in a purity of CD4+ T cells from about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 99%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 99%, about 55% to about 100%, about 60% to about 65%, about 60% to about 70%, about 60% to about 75%, about 60% to about 80%, about 60% to about 85%, about 60% to about 90%, about 60% to about 95%, about 60% to about 99%, about 60% to about 100%, about 65% to about 70%, about 65% to about 75%, about 65% to about 80%, about 65% to about 85%, about 65% to about 90%, about 65% to about 95%, about 65% to about 99%, about 65% to about 100%, about 70% to about 75%, about 70% to about 80%, about 70% to about 85%, about 70% to about 90%, about 70% to about 95%, about 70% to about 99%, about 70% to about 100%, about 75% to about 80%, about 75% to about 85%, about 75% to about 90%, about 75% to about 95%, about 75% to about 99%, about 75% to about 100%, about 80% to about 85%, about 80% to about 90%, about 80% to about 95%, about 80% to about 99%, about 80% to about 100%, about 85% to about 90%, about 85% to about 95%, about 85% to about 99%, about 85% to about 100%, about 90% to about 95%, about 90% to about 99%, about 90% to about 100%, about 95% to about 99%, about 95% to about 100%, or about 99% to about 100%.

In some cases, the methods described herein result in a purity of CD4+ T cells that can be 2×, 3×, 4×, 5×, 6×, 7×, 8×, 9×, or 10× greater than a purity of CD4+ T cells obtained from a conventional cell separation method of a negative control.

The methods described herein may separate target T cells from other white blood cells of a sample and result in greater recovery of target T cells compared to that of conventional methods or negative controls (e.g., no binding agent). A recovery of target T cells can be measured as a fraction of the target T cells from the original input sample. In some cases, the methods described herein result in a recovery value of CD4+ T cells of at least about 0.1, at least about 0.15, at least about 0.2, at least about 0.25, at least about 0.3, at least about 0.35, at least about 0.4, at least about 0.45, at least about 0.5, at least about 0.55, at least about 0.6, at least about 0.65, at least about 0.7, at least about 0.75, at least about 0.8, at least about 0.85, at least about 0.9, or greater than about 0.9. In some cases, the methods described herein result in a recovery value of CD4+ T cells of at most about 0.9, at most about 0.85, at most about 0.8, at most about 0.75, at most about 0.7, at most about 0.65, at most about 0.6, at most about 0.55, at most about 0.5, at most about 0.45, at most about 0.4, at most about 0.35, at most about 0.3, at most about 0.25, at most about 0.2, at most about 0.15, at most about 0.1, or less than about 0.1. In some cases, the methods described herein result in a recovery value of CD4+ T cells from about 0.1 to about 0.8. In some cases, the methods described herein result in a recovery value of CD4+ T cells from about 0.1 to about 0.15, about 0.1 to about 0.2, about 0.1 to about 0.25, about 0.1 to about 0.3, about 0.1 to about 0.35, about 0.1 to about 0.4, about 0.1 to about 0.45, about 0.1 to about 0.5, about 0.1 to about 0.6, about 0.1 to about 0.7, about 0.1 to about 0.8, about 0.15 to about 0.2, about 0.15 to about 0.25, about 0.15 to about 0.3, about 0.15 to about 0.35, about 0.15 to about 0.4, about 0.15 to about 0.45, about 0.15 to about 0.5, about 0.15 to about 0.6, about 0.15 to about 0.7, about 0.15 to about 0.8, about 0.2 to about 0.25, about 0.2 to about 0.3, about 0.2 to about 0.35, about 0.2 to about 0.4, about 0.2 to about 0.45, about 0.2 to about 0.5, about 0.2 to about 0.6, about 0.2 to about 0.7, about 0.2 to about 0.8, about 0.25 to about 0.3, about 0.25 to about 0.35, about 0.25 to about 0.4, about 0.25 to about 0.45, about 0.25 to about 0.5, about 0.25 to about 0.6, about 0.25 to about 0.7, about 0.25 to about 0.8, about 0.3 to about 0.35, about 0.3 to about 0.4, about 0.3 to about 0.45, about 0.3 to about 0.5, about 0.3 to about 0.6, about 0.3 to about 0.7, about 0.3 to about 0.8, about 0.35 to about 0.4, about 0.35 to about 0.45, about 0.35 to about 0.5, about 0.35 to about 0.6, about 0.35 to about 0.7, about 0.35 to about 0.8, about 0.4 to about 0.45, about 0.4 to about 0.5, about 0.4 to about 0.6, about 0.4 to about 0.7, about 0.4 to about 0.8, about 0.45 to about 0.5, about 0.45 to about 0.6, about 0.45 to about 0.7, about 0.45 to about 0.8, about 0.5 to about 0.6, about 0.5 to about 0.7, about 0.5 to about 0.8, about 0.6 to about 0.7, about 0.6 to about 0.8, or about 0.7 to about 0.8.

In some cases, the methods described herein result in a recovery of CD4+ T cells that can be 2×, 3×, 4×, 5×, 6×, 7×, 8×, 9×, or 10× greater than a recovery of CD4+ T cells obtained from a conventional cell separation method of a negative control.

### Rapid Immunophenotyping of Cell Biomarkers

Rapid immunophenotyping methods generally use fluorescent antibody dyes to label cell biomarkers, where each biomarker is labeled with a different fluorescent dye that emits a specific wavelength of light (i.e. different color). Microscopic imaging and computer vision can be used to count the distinct fluorescent labels and determine the number of biomarkers in the population.

In conventional systems, the number of biomarkers that can be used simultaneously in the analysis is limited (e.g., to a maximum of 5 biomarkers), because the fluorescent emission spectrum is relatively narrow. The probability that two dye light emissions overlap increases with each new dye that is added to the system. After only a few dyes being added to the system, the overlap is too great to identify distinct labels. A system that can accurately count more a greater number of biomarkers simultaneously provides several benefits to laboratory analyses, including cell therapy manufacturing.

As described herein, the shortfalls of conventional systems can be overcome by adding fluorescent dyes serially in between repeated isolation steps via inertial sorting. This approach allows for an unlimited number of biomarkers to be measured on the same sample, and also can reduce the number of fluorescent dyes that are needed in the system. The process and design described herein can provide, for example, for automated, closed-system, high throughput biomarker counting and cell viability analysis.

Rapid immunophenotyping of cell biomarkers by inertial sorting can be implemented in various steps. For example, a sample (e.g., a mixed aliquot of cells in solution) can be input into a cartridge (e.g., a microfluidic cartridge) that pushes the solution through a series of microfluidic channels at a high rate of flow (Input step). A fluorescent antibody dye can be mixed into the microfluidic channel and bind with the cells that express that specific biomarker targeted by the antibody (Labeling step). A series of microfluidic channels can bifurcate the solution between the fluorescently-labeled and the unlabeled cells using, for example, inertial lift (Isolation step). Content of the channel with labeled cells can be sent to an imaging chamber on the device (or off the device) (Collection step). Content of the channel(s) with unlabeled cells can be sent to another cartridge, for example.

The steps described above can be repeated for a new biomarker. The new biomarker can be labeled with the same fluorophore, because the population of cells labeled in the prior process have already been separated from the cell population to be processed. In this case, the only change would be the antibody that is conjugated with the fluorescent dye. This process can be repeated infinitely and allows for continual isolation and collection of cell populations with a specific biomarker.

In some implementations, the device outputs one channel for each biomarker and one channel for the unlabeled solution (#biomarkers+1). Each output can be sent to an imaging chamber (either on the device or on a different device). Two additional fluorescent dyes can be added to the imaging chamber to determine the count and viability of the output's cell population (Imaging and computer vision).

The microfluidic design can be the same for each cartridge, which provides for ease of manufacturing and compatibility. The fluorescent antibody dye can be added in any order (provided that the device keeps track of the order). A single fluorophore (e.g., a fluorophore with an emission wavelength of about 386 nm, 410 nm, 442 nm, 445 nm, 455 nm, 478 nm, 480 nm, 483 nm, 510 nm, 516nm, 533 nm, 545 nm, 555 nm, 570 nm, 578 nm, 580 nm, 596 nm, 602 nm, 613 nm, 615 nm, 617 nm, 620 nm, 639 nm, 647 nm, 660 nm, 668 nm, 670 nm, 690 nm, 694 nm, 695 nm, 702 nm, or 770 nm, or any range between these values, or a fluorophore with an absorption wavelength of about 325 nm, 360 nm, 345 nm, 350 nm, 430 nm, 480 nm, 490 nm, 495 nm, 530 nm, 535 nm, 547 nm, 550 nm, 556 nm, 565 nm, 560 nm, 565 nm, 757 nm, 580 nm, 590 nm, 615 nm, 621 nm, 650 nm, 663 nm, 675 nm, 679 nm, or 743 nm, or any range between these values) can be used for all cartridges-only the conjugated antibody would vary (depending on the biomarker of interest). In some implementations, very few (e.g., three) fluorescent filters need to be used in the optics of the imaging chamber. Cartridges can be serially connected, where each new cartridge in the pipeline can label a different antibody. The microfluidic chamber can be designed to allow for washing and concentrating the solutions.

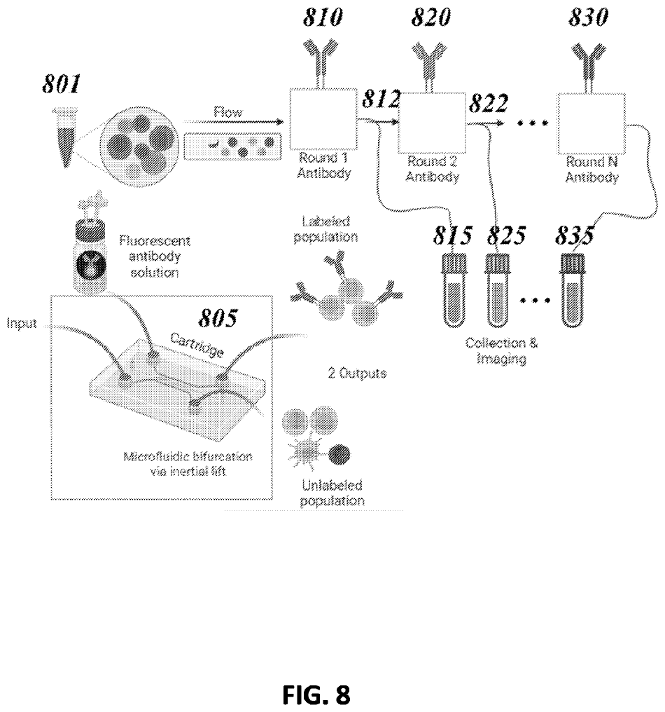

FIG. 8 shows an exemplary schematic of a method for rapid immunophenotyping of cell biomarkers by inertial sorting. The method can be implemented into a cartridge (e.g., a microfluidic cartridge), for example. A sample 801 comprising cells in a solution can be input into a cartridge 805, where the solution flows through a series of channels at a high flow rate. Cells can be labeled in a channel by introducing a first fluorophore-conjugated binding agent 810 (e.g., fluorophore-conjugated antibody) into the channel. Following the labeling of cells with the first fluorophore-conjugated binding agent 801, a series of microfluidic channels can bifurcate the solution between the cells using inertial lift. Cells labeled with the fluorophore-conjugated binding agent 801 can be directed to an imaging chamber 815, on or off the device, while unlabeled cells can be directed to a channel 812 where cells are labeled with a second fluorophore-conjugated binding agent 820 (e.g., fluorophore-conjugated antibody) introduced into the channel. Following the labeling of cells with the second fluorophore-conjugated binding agent 820, a series of microfluidic channels can efficiently bifurcate the solution between the cells again using inertial lift. Cells labeled with the second fluorophore-conjugated binding agent 820 can be directed to an imaging chamber 825, on or off the device, that is specific for the second fluorophore, and unlabeled cells can be directed to a channel 822 where further labeling can occur. Iterative rounds of labeling and bifurcating labeled and unlabeled cells using inertial lift can allow for labeling (and analysis) of cells with an arbitrary number of fluorophore-conjugated binding agents 830 provided they are in combination with suitable imaging chambers 835 specific for the fluorophore conjugated to the binding agent.

### Computer Control Systems

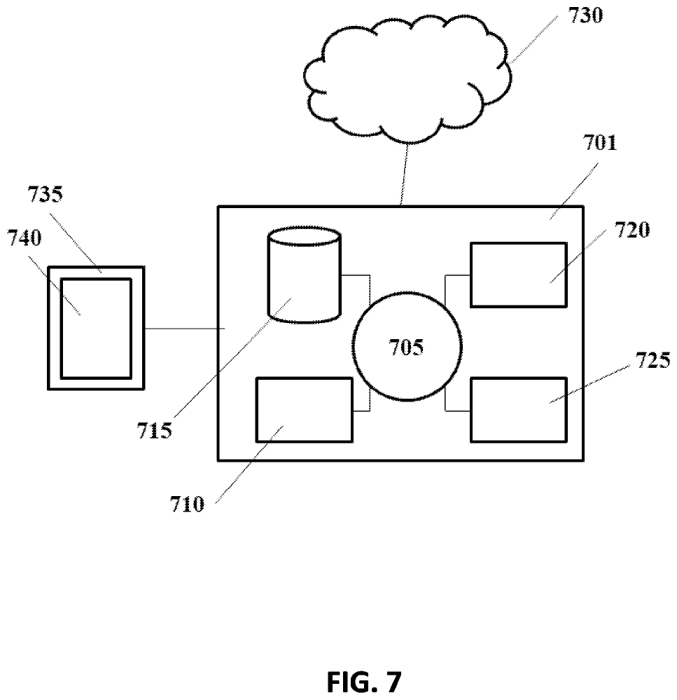

The present disclosure provides computer control systems that are programmed to implement methods of the disclosure. FIG. 7 shows a computer system 701 that is programmed or otherwise configured to perform processes described throughout this disclosure. The computer system 701 can regulate various aspects of systems of the present disclosure, such as, for example, add reagents to initiate polymerization or dissolution of beads or polymer matrices, initiate the addition or incubation of cells in a reaction chamber, or activate a plunger. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which can enable devices coupled to the computer system 701 to behave as a client or a server.

The CPU 705 can execute a sequence of machine-readable instructions, which can be embodied in a program or software. The instructions can be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., laboratory technician). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine-readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 701 can include or be in communication with an electronic display 735 that comprises a user interface (UI) 740 for providing, for example, status reports of the system, data relating to the characteristic of the cells. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705.

### EXAMPLES

### Example 1. Isolation of Cells Using Size-Based Selection

In this example, a sample (see e.g., FIG. 1) including cells 101 and cells 105 is provided. Beads 110 that are able to bind cells 105 are added and incubated with the sample. Upon incubation, cells 101 remain unbound whereas cells 105 bind to the beads 110 to form bead-cell complexes 120. The solution then flows through a separation filter 130 with pores that are smaller than the bead 110 (and bead-cell complexes 120). The beads 110 and bead-cell complexes 120 are unable to pass through the pore, whereas any unbound cells 101 pass through.

### Example 2. Isolation of Cells Using Dissolvable Bead Columns

In this example, beads (see e.g., FIG. 4) 410 are composed of dissolvable polymer and include a binding agent that can bind to a cell of interest. The beads are packed into a column. The beads are packed such that a solution of cells can flow through the column and pass by the beads. At the bottom of the column, a membrane or filter 420 prevents the beads from passing through while allowing cells to flow through. A sample 430 including cells 432 and cells 434 is added to the column and contacts at least some of the beads 410. The beads 410 include binding agents that are able to bind to cells 434, but do not bind to cells 432. As the cells flow through the column and contact beads 410, cells 434 may be bound to beads 410 via the binding agent forming bead-cell complexes 435. Cells 432, which do not bind to beads 410, such as cells 432, pass through the filter 420 and exit the column. After the bead-cell complexes 435 are formed, a wash solution 440 can be added to the column to remove any contaminants or residual material that are present in the column. Based at least on pH level, ionic strength or other chemical characteristics, the wash solution used can affect the purity and yield for isolated and/or selected cells. For example, a higher ionic concentration may disrupt non-specific interactions and result in a more stringent wash condition. Similarly, pH levels that are higher or lower than a neutral pH can alter interactions and allow for a more stringent wash condition. Additionally, the speed at which the wash solution is applied, as well as the agitation of the wash solution, can modulate the purity and yield. Stringent wash conditions can reduce overall yield and can improve purity, whereas less stringent wash conditions can result in a higher overall yield and reduce purity. The cell-bead complexes 435 remain in the column. Cells 432 are eluted out of the column. The beads 410 of the bead-cell complexes 435 can be dissolved by the addition of a dissolution reagent 450 thereby releasing cells 434, which can flow through the filter 420 out of the column, for example, to be harvested or otherwise directed to another processing step.

### Example 3. Use of Magnetic Dissolvable Beads

In this example, a sample of cells, including cells 510, is collected from a subject. Dissolvable magnetic beads (see e.g., FIGS. 5A-5C) 501 can be generated using a microfluidic droplet generator, and a solution including alginate, magnetic particles, and antibodies. The resulting dissolvable magnetic bead 501 includes alginate 502, magnetic particles 505, and antibody 508. The dissolvable magnetic beads 501 are mixed with the sample and cells 510 may bind with the dissolvable magnetic beads 501 forming bead-cell complexes 515. The bead-cell complexes 515 (and unbound dissolvable beads 501) can be separated using a magnetic force that attracts the bead-cell complexes 515 (and unbound dissolvable beads 501). Upon separation of the bead-cell complexes 515 (and unbound dissolvable beads 501), a wash solution can be applied to remove contaminants and residual non-specific binding. Based at least on pH level, ionic strength or other chemical characteristics, the wash solution used can affect the purity and yield for isolated and/or selected cells. For example, a higher ionic concentration may disrupt non-specific interactions and result in a more stringent wash condition. Similarly, pH levels that are higher or lower than a neutral pH can alter interactions and allow for a more stringent wash condition. Additionally, the speed at which the wash solution is applied, as well as the agitation of the wash solution, can modulate the purity and yield. Stringent wash conditions can reduce overall yield and can improve purity, whereas less stringent wash conditions can result in a higher overall yield and reduce purity. To release cells 510, a dissolving reaction can be performed by adding citrate or other reagent to dissolve dissolvable beads 501 of the bead-cell complexes 515. During dissolution magnetic particles 505 and cells 510 are released into the solution. The solution including the magnetic particles 505 and cells 510 can then be subjected to a magnetic force to attract the magnetic particles 505 and remove the magnetic particles 505 from the solution while cells 510 remain. The magnetic particles 505 can then be discarded or recycled to generate new magnetic dissolvable beads.

### Example 4. Dead-End Filtration Selection of T-Cells

In this example, a sample of cells, including cells 601, is collected from a subject. The sample is added into a microfluidic device for separation, isolation, and/or selection of cells. A buoyant bead (e.g., 2-20 μm) is functionalized with streptavidin and is attached to a biotinylated anti-CD3 Fab fragment to generate a buoyant bead 610 that is able to bind CD3 (see e.g., FIG. 6A). The buoyant beads 610 are added to a reaction chamber 622 (see e.g., FIG. 6B) of the microfluidic device. In configuration 620, the reaction chamber 622 includes beads 610. The microfluidic device can include a plunger 621 that can modulate the size of the active volume of the reaction chamber 622 and apply pressure to the liquid (e.g., buffer, media, or another solution) to push the liquid out of the reaction chamber 622. In configuration 620, the plunger 621 is not activated. The microfluidic device can include a filter 625 (e.g., 5-20 μm pore size) located at the bottom of the reaction chamber 622. The pores of the filter 625 are smaller than the size of a buoyant bead 610, thereby preventing buoyant beads 610 to pass through, while allowing unbound cells to pass through. The microfluidic device has an input channel 623, a wash channel 624, an output channel 626 and a recirculation channel 627. The input channel 623, and the wash channel 624 can run parallel to one another and can be in fluidic communication with the filter 625 disposed between them to allow liquid to move freely between the two channels but prevent the movement of cell/bead complexes between the channels. The sample including the cells can be added to the reaction chamber through the input channel 623 and recirculation channel 627, and interact with the buoyant beads 610 to form bead-cell complexes 615. As shown in configuration 630, the plunger 621 can be activated to push the liquid through the filter 625 while bead-cell complexes 615 (and unbound buoyant beads 610) remain above the filter 625. The buoyant properties of the bead-cell complexes 615 (and unbound buoyant beads 610) may prevent or reduce clogging of the filter 625. Unbound cells can exit the microfluidic device through the output channel 626 as denoted by 635. The plunger 621 can then be returned to its starting position resetting the active volume of the reaction chamber 622, as shown in configuration 640. New liquid (e.g., sample, wash buffer, or elution buffer) can be added to the reaction chamber through the input channel 623 and recirculation channel 627 as denoted by 645. For example, an elution buffer can be added into the reaction chamber 622 which is configured to disrupt the bead-cell complexes 615 resulting in unbound cells 601 and buoyant beads 610. As shown in configuration 650, after elution of the cells 601, the plunger 621 can be activated thereby pushing cells 601 through the filter 625 for collection or further processing. The buoyant properties of the beads 610 may reduce or prevent clogging of the filter 625, and the size of the beads 610 may prevent from passing through the filter 625 to avoid interference with downstream processes or products.

### Example 5. Rapid Immunophenotyping of Cell Biomarkers by Inertial Sorting

In this example, inertial lift-based cell sorting (e.g., in a microfluidic device) is utilized to overcome limitations associated with labeling cell types with multiple fluorophore-conjugated binding agents which may have spectrum overlap and limit the number of cellular antigens that can be analyzed in a given pool of cells using fluorophore-conjugated binding agents. This approach can allow for an arbitrary number of cell markers with fluorophore-conjugated binding agents.

In this example, with reference to FIG. 8, the method first involves inputting a sample 801 comprising cells in solution into a cartridge 805 (e.g., a microfluidic cartridge) that pushes the solution through a series of channels at a high rate of flow. Cells can then be labeled in a channel by introducing a first fluorophore-conjugated binding agent 810 (e.g., fluorophore-conjugated antibody) into the channel. Following the labeling of cells with the first fluorophore-conjugated binding agent 801, a series of microfluidic channels can efficiently bifurcate the solution between the cells using inertial lift. Cells labeled with the fluorophore-conjugated binding agent 801 can be directed to an imaging chamber 815, on or off the device, while unlabeled cells can be directed to a channel 812 where cells are labeled with a second fluorophore-conjugated binding agent 820 (e.g., fluorophore-conjugated antibody) introduced into the channel. Following the labeling of cells with the second fluorophore-conjugated binding agent 820, a series of microfluidic channels can efficiently bifurcate the solution between the cells again using inertial lift. Cells labeled with the second fluorophore-conjugated binding agent 820 can be directed to an imaging chamber 825, on or off the device, that is specific for the second fluorophore, and unlabeled cells can be directed to a channel 822 where further labeling can occur. Iterative rounds of labeling and bifurcating labeled and unlabeled cells using inertial lift can allow for labeling (and analysis) of cells with an arbitrary number of fluorophore-conjugated binding agents 830 provided they are in combination with suitable imaging chambers 835 specific for the fluorophore conjugated to the binding agent.

### Example 6. Separation of Target T Cells From Non-Target White Blood Cells

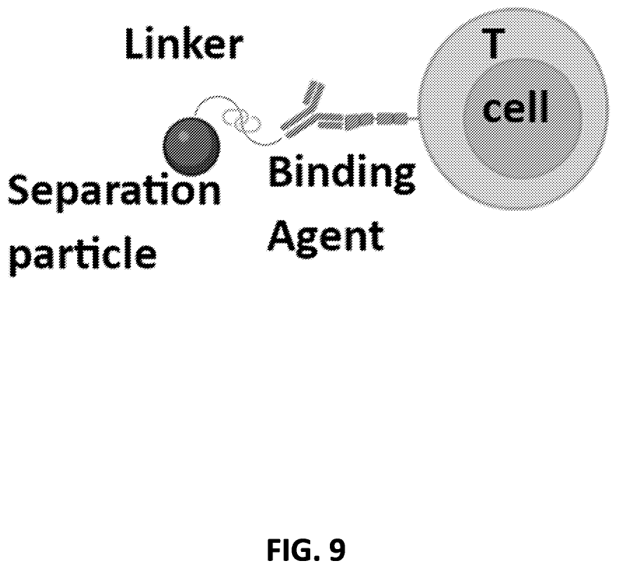

In this example, multiple cell separation methods were assessed to separate target CD4+ T cells from background white blood cells of a sample. Separation of target T cells from the background of other white blood cells utilized a separation particle coupled with a linker coupled to a binding agent that bound to a target surface antigen on the target T cell (FIG. 9). A summary of separation particles and combination methods is shown in Table 1. Performance was evaluated in terms of yield, purity, and viability of target T cells.

TABLE 1
Summary of separation methods

| | Separation | Linker/Binding | Yield | Purity | Viability |
|---|---|---|---|---|---|
| | Particle | Agent | (Threshold >50%) | (Threshold >80%) | (Threshold >90%) |
| A | Magnetic | Biotin/RPA-T4 | 51-62 | 82-90 | 92-97 |
| | nanoparticle coated | IgG | | | |
| | with Streptavidin | | | | |
| B | Magnetic | Recombinant | 69-82 | 90-92 | 97 |
| | nanoparticle coated | Biotin | | | |
| | with Streptavidin | analogue/Fab | | | |
| | | CD4 | | | |
| C | Magnetic | Biotin/OKT-4 | 67 | 93 | 88 |
| | nanoparticle coated | IgG | | | |
| | with Streptavidin | | | | |
| D | Polystyrene Bead | Biotin/OKT4 | >50 | >80 | |
| | for inertial size- | IgG | | | |
| | based separation | | | | |
| E | Lipid microbubble | Biotin/RPA-T4 | 20-40 | 70-97 | 72-87 |
| | (5-10 microns) for | IgG | | | |
| | buoyancy | | | | |
| | separation | | | | |
| F | Lipid microbubble | Biotin/IgG | 22-44 | 74-97 | 87-96 |
| | (<5 microns) for | | | | |
| | buoyancy | | | | |
| | separation | | | | |
| G | Magnetic | Dissolvable | >40 | >70 | >80 |
| | microparticle | polymer | | | |
| | coated with | matrix/RPA-T4 | | | |
| | dissolvable | IgG | | | |
| | polymer matrix | | | | |

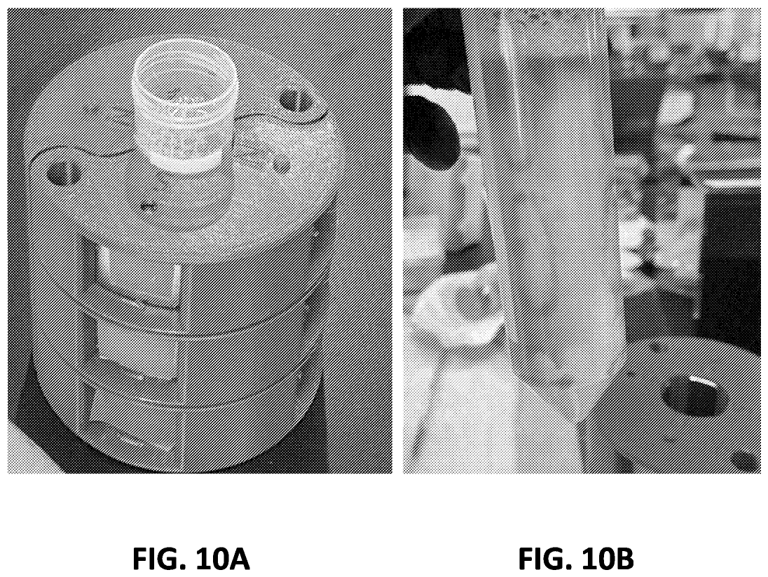

For the methods using the magnetic nanoparticle (see A-C in Table 1), cells were incubated/labeled with the nanoparticles and the separation was performed in an open gradient quadrupole magnet in a 50 mL batch tube configuration (FIG. 10A). After 5 minutes of incubation, cells labeled with magnetic nanoparticles formed rings on the tube sidewall corresponding to the high magnetic field gradient regions between the north and south magnetic poles (FIG. 10B). Non-target cells in the supernatant were aspirated, then 50 mL of wash buffer was added and used to resuspend the cells. The separation/washing was repeated to further remove non-target cells and finally the target cells were eluted by resuspending in suitable volume of elution buffer (e.g. 10 mL) while removing the tube from the magnet.

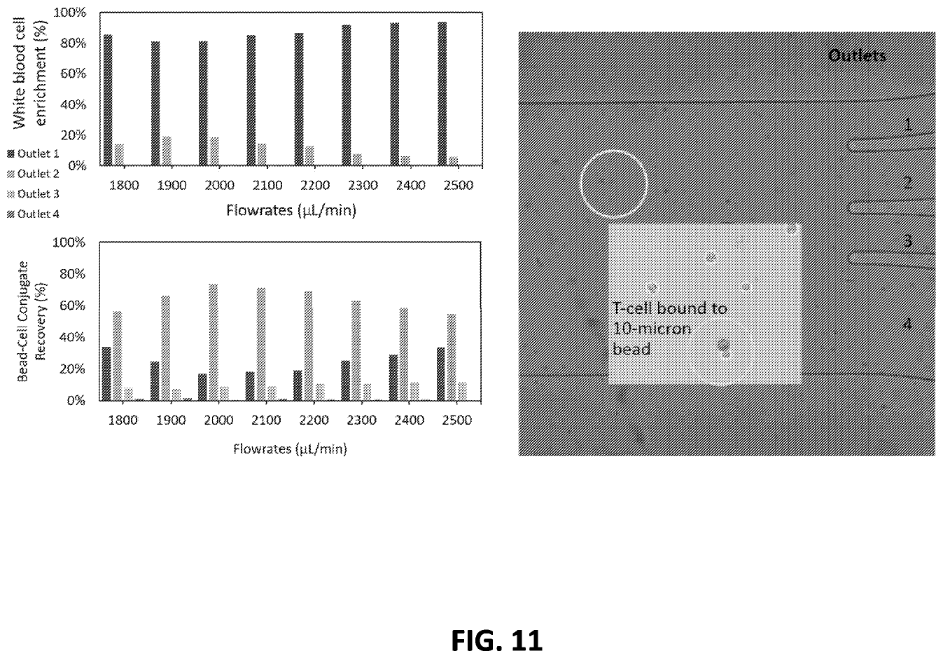

For polystyrene bead size-based inertial separation (see D in Table 1), cells were incubated/labeled with the beads and separation was performed by passing at high flow rate though a microfluidic inertial focusing chip. The inertial forces focused the cells and cell/bead populations to distinct streamlines which were collected in one of the four outlets of the chip. Specifically, the unlabeled non-target white blood cells were enriched in outlet 1, while the target bead-cell conjugate population was recovered in outlet 2 (FIG. 11).

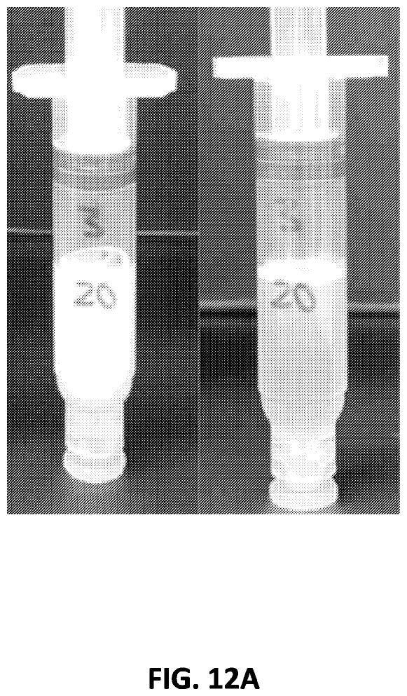

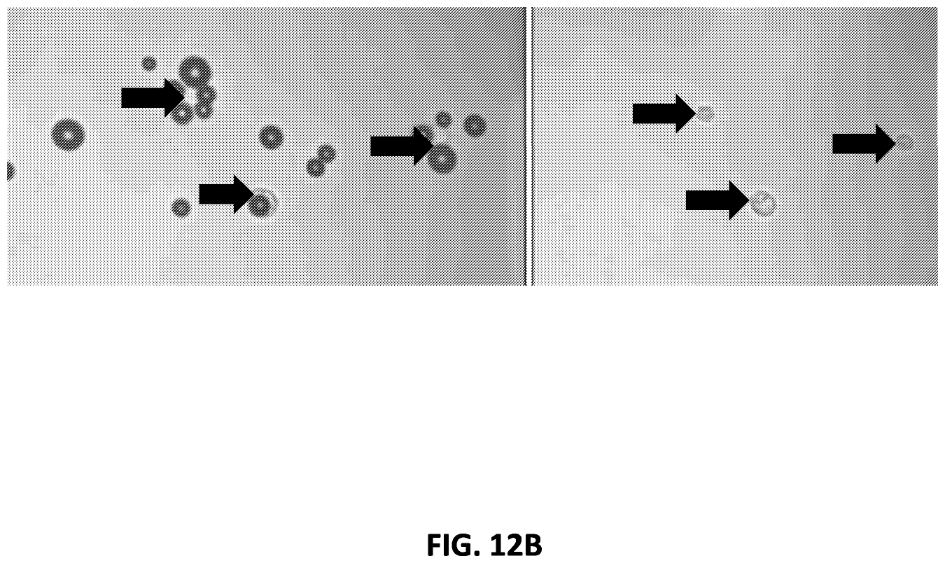

For methods utilizing lipid microbubble separation (see E-F in Table 1), cells were incubated/labeled with the microbubbles and separation was performed by gravity settling for 20 minutes (or by centrifugation at 100 g for 1 minute). The reaction chamber was typically a syringe to allow for easy removal of non-target cells that settled to the bottom (FIG. 12A). After flushing out of non-target cells, wash buffer was added and separation was repeated to increase purity. To enable sequential cell separation, the separation particle was removed or the link between the particle and the binding agent was broken. For lipid microbubbles, 2 bar of pressure was used for 1-2 minutes to collapse/dissolve the bubble (FIG. 12B).

For the magnetic nanoparticle with the recombinant biotin linker (C in Table 1), the link was broken by adding an excess of biotin which outcompeted the lower affinity recombinant biotin in the streptavidin binding site. Results are shown in Table 2. The “CD4 Isolate” fraction after magnetic separation with the magnetic nanoparticle was incubated in 1 mM biotin for 10 mins at room temperature. Magnetic separation was then performed again to capture the dissociated particles. The remaining cells in the supernatant were then stained/counted to represent the “Dissociated CD4” fraction, showing a 77% yield of the original CD4 Isolate fraction.

TABLE 2
Results of cell separation using magnetic
nanoparticle with recombinant biotin linker

| | Viability | Purity CD4% | Monocytes | Yield |
|---|---|---|---|---|
| PBMC | 65% | 60% | 6% | |
| CD4 Isolate | 69% | 92% | 3% | 82% |
| Dissociated CD4 | 77% | 93% | 2% | 77% |

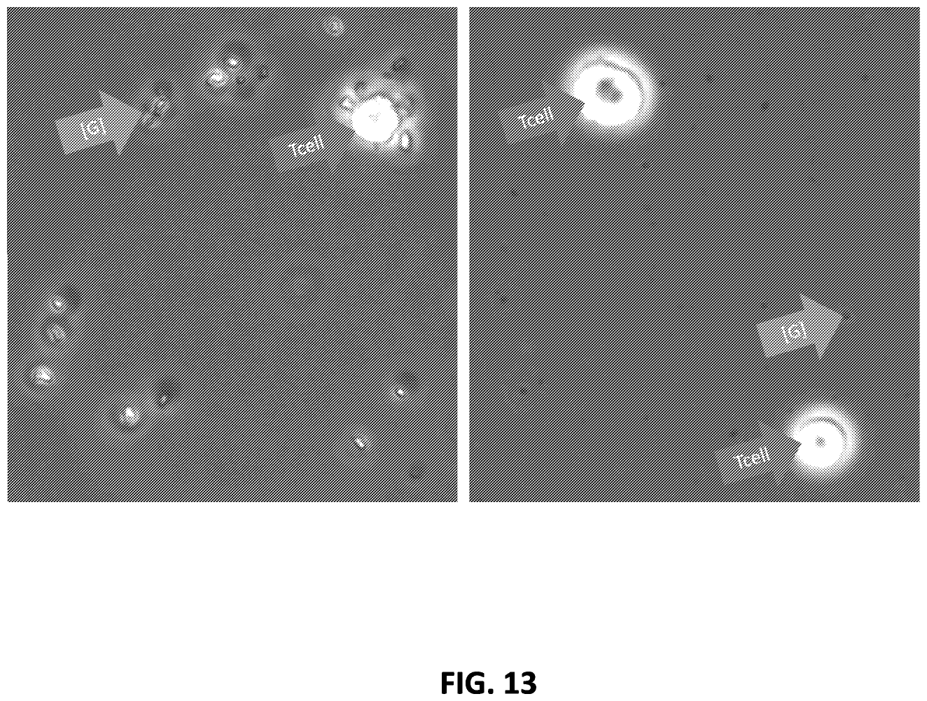

For magnetic microparticle coated with dissolvable polymer matrix (see G in Table 1), the link was broken by adding a reducing agent to dissolve the polymer matrix shell. TCEP (tris(2-carboxyethyl)phosphine) was used in this study. A T cell bound to magnetic microparticles coated with dissolvable polymer matrix was imaged along with free-floating particles not bound to the T cell due to the excess of beads as compared to T cells (FIG. 13). In the left panel of FIG. 13, the polymer matrix shell was clearly visible as a dark halo surrounding the magnetic core particles. The right panel of FIG. 13 shows cells after addition of TCEP and incubating for 10 minutes. The polymer matrix was now dissolved after the TCEP treatment, leaving behind only the core magnetic particles. The T cells dissociated from the magnetic particles and were no longer bound to the particles which were subsequently removed by an additional magnetic purification step.

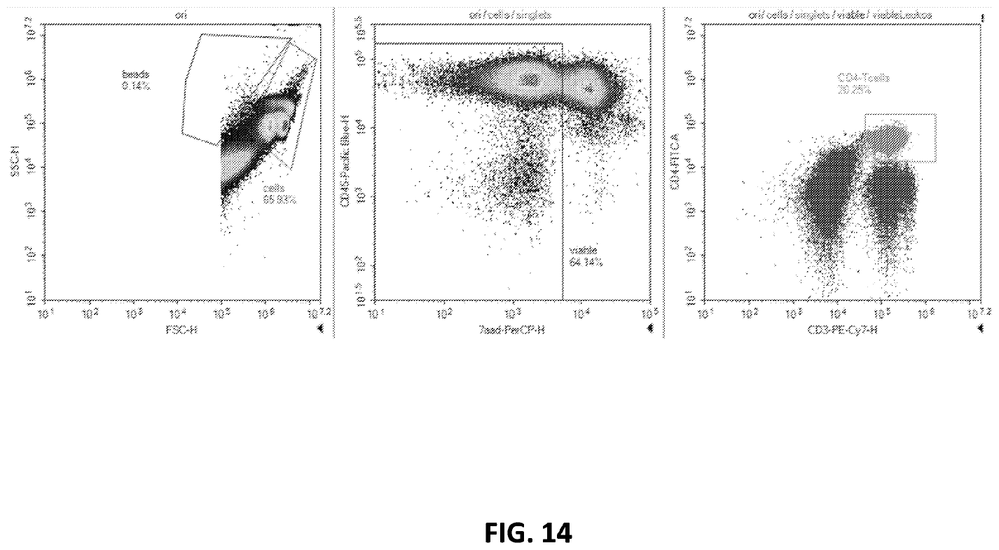

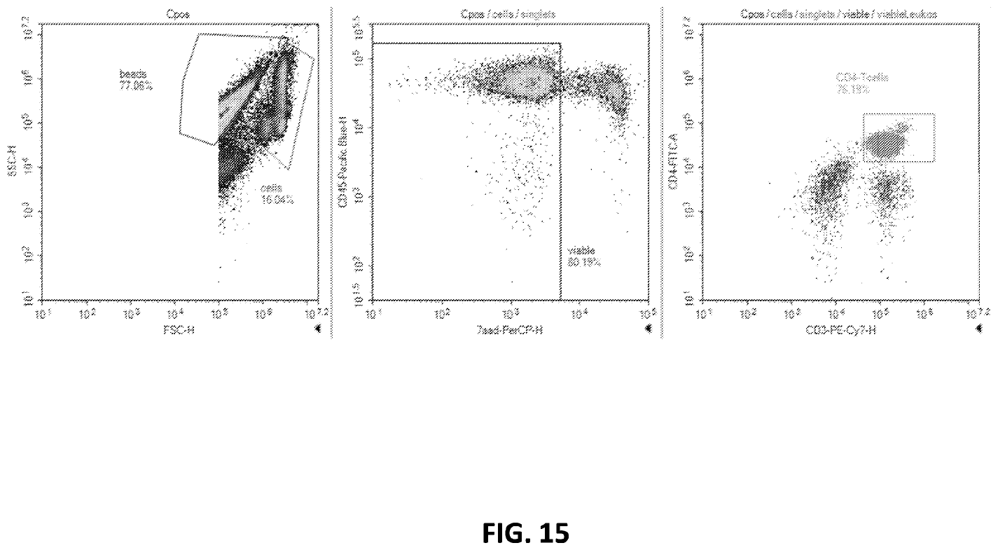

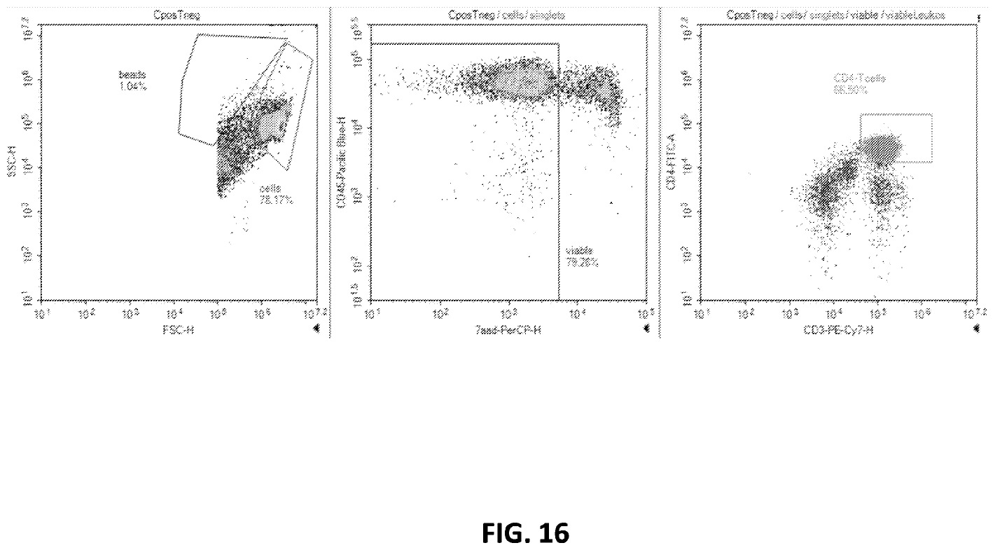

The performance of the separation to purify the target CD4+ T cells bound by the RPA-T4 IgG conjugated to the polymer on the magnetic particles was assessed by flow cytometry. As shown in the flow cytometry plots of FIG. 14, the original peripheral blood mononuclear cells (PBMCs) input sample was gated on scatter to identify the bead and cell populations and stained with CD45, CD3, CD4 antibodies and 7-AAD viability dye to identify the target viable CD4+ T cells. The flow cytometry plots of FIG. 15 show the purified CD4+ target cells immediately following magnetic separation with the magnetic microparticle coated with dissolvable polymer matrix (e.g., beads). The beads were clearly visible in the scatter plot. The scatter profile of the cells was altered as a result of binding to the beads. The flow cytometry plots of FIG. 16 show the purified CD4+ Target cells after dissolution of the beads with TCEP and removal of residual free unbound magnetic particles. The beads were removed from the scatter gate and also the cells restored their original scatter profile showing the effectiveness of TCEP to dissolve the matrix and dissociate the beads from the cells.

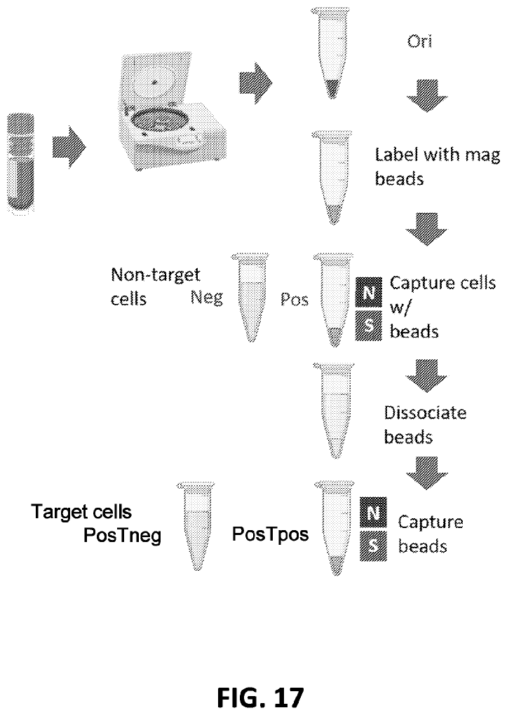

The purity and recovery for the separation of CD4+ T cells was assessed following separation (FIG. 17). Purity and recovery were tested in three technical replicates compared to a negative control bead (no RPA-T4 IgG antibody conjugated to the polymer matrix shell of the magnetic bead). Frozen PBMCs were the input sample. These were washed two times (PBS, 2 mM EDTA, 0.5% BSA), 10e6/mL with approximately 60% viable ori fraction. Next, three technical replicates were performed with the negative control. There were 10e6 cells with 50e6 beads in 200 μL. These were mixed for 30 minutes at room temperature on the rotator. Buffer was added to 1 mL and cells were then incubated on the magnet for 5 minutes. The supernatant was then removed, designating the “neg” fraction. The pellet was then resuspended in 1 mL, designating the “pos” fraction. TCEP was then added to dissociate the beads. The mixture was incubated for 10 minutes, and then incubated for 5 minutes on the magnet. The supernatant was removed, designating the “posTneg” fraction. Finally, the pellet was resuspended in 1 mL, designating the “posTpos” fraction.

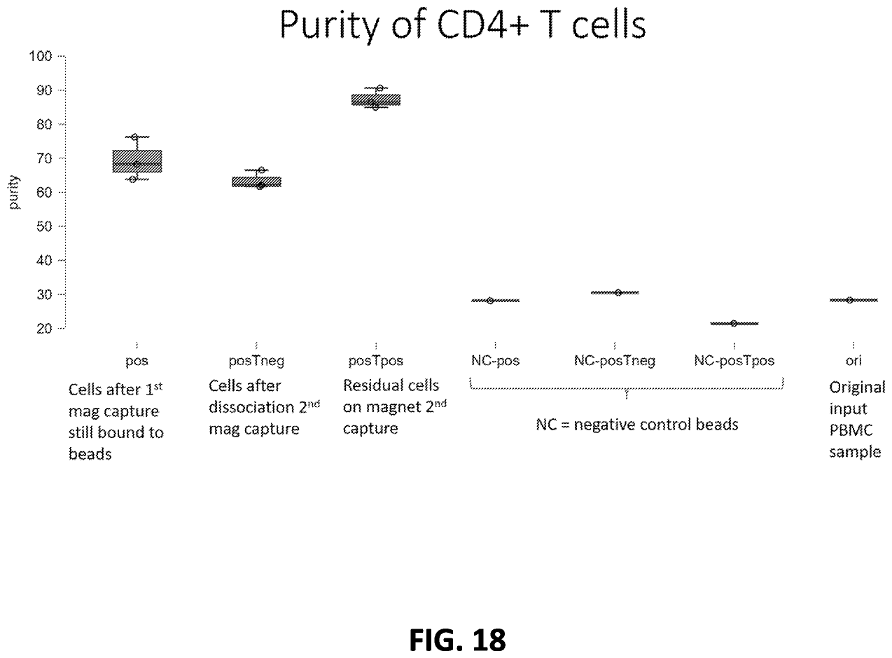

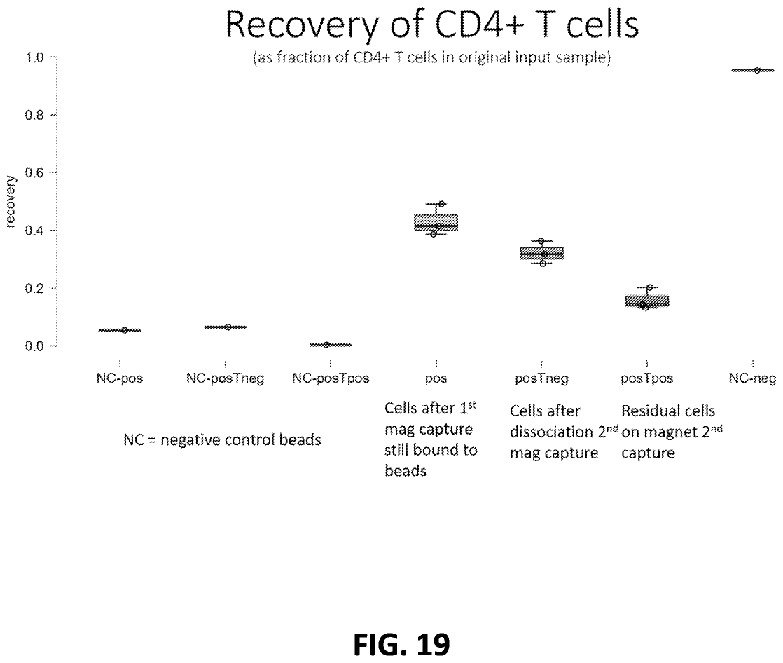

The purity of the cells were compared to the negative control groups. The “pos” fraction, “posTneg” fraction, and “posTpos” fraction all showed greater purity than their corresponding negative control group (FIG. 18). The recovery of the cells were also compared to the negative control groups. Recovery was measured as a fraction of the CD4+ T cells in the original input sample. The “pos” fraction, “posTneg” fraction, and “posTpos” fraction all showed greater recovery of CD4+ T cells than their corresponding negative control group.

While preferred embodiments of the present disclosure have been shown and described herein, it will be obvious to those skilled in the art that such embodiments are provided by way of example only. It is not intended that the invention be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the invention and that methods and structures within the scope of these claims and their equivalents be covered thereby.

What is claimed is:

1. A method of selecting cells, the method comprising:
a) providing a plurality of cells and a plurality of beads, wherein at least one bead of the plurality of beads comprises a binding agent capable of binding a subset of the plurality of cells;
b) subjecting the plurality of cells and plurality of beads to conditions to allow the plurality of beads to bind the subset of the plurality of cells, thereby generating (i) at least one bead-cell complex, and (ii) at least one unbound cell, wherein the at least one bead-cell complex is a larger size compared to the at least one unbound cell;
c) subjecting the at least one bead-cell complex and the at least one unbound cell to a size separation, thereby separating the at least one bead-cell complex from the at least one unbound cell.

2. The method of claim 1, wherein the size separation comprises use of a filter.

3. The method of claim 2, wherein a size of pores of the filter is less than a size of a bead-cell complex and greater than a size of the at least one unbound cell.

4. The method of claim 1, wherein the size separation comprises at least one of inertial focusing and deterministic lateral displacement.

5. The method of claim 1, wherein the plurality of beads comprises buoyant beads.

6. The method of claim 5, wherein the size separation comprises use of a filter and pushing the at least a subset of the unbound cells through the filter.

7. The method of claim 1, further comprising, subsequent to c), collecting separated unbound cells.

8. The method of claim 1, further comprising, subsequent to c), collecting at least one separated bead-cell complex.

9. The method of claim 8, further comprising, subsequent to the collecting, subjecting the at least one separated bead-cell complex to at least one release condition, thereby releasing the subset of the plurality of cells from the plurality of beads.

10. The method of claim 9, wherein the release condition comprises a high salt concentration solution.

11. The method of claim 9, wherein the plurality of cells are immersed in a medium, and wherein the release condition comprises a change in the pH of the medium.

12. The method of claim 11, wherein the change in pH comprises an increase of pH.

13. The method of claim 11, wherein the change in pH comprises a decrease of pH.

14. The method of claim 11, wherein the medium is a separation buffer.

15. The method of claim 11, wherein the pH is changed from physiological to a pH value above about 8.

16. The method of claim 9, wherein the release condition comprises a biotin solution.

17. The method of claim 16, wherein the biotin solution comprises a desthiobiotin-based binding agent.

18. The method of claim 16, wherein the biotin solution comprises a recombinant biotin having a binding 1 affinity lower than native biotin.

19. The method of claim 1, wherein the binding agent and the bead are linked via a linker.

20. The method of claim 19, wherein the linker comprises a biotin and streptavidin.

21. The method of claim 19, wherein the linker comprise a covalent linker.

22. The method of claim 19, wherein the linker is generated by (i) a bead comprising a first reactive group and (ii) a binding agent comprising a second reactive group and reacting the first reactive group with the second reactive group to form the linker.

23. The method of claim 19, wherein the linker is generated by (i) a bead comprising a first binding member and (ii) a binding agent comprising a second binding member and reacting the first binding member with the second binding member to form the linker.

24. The method of claim 23, wherein the first binding member comprises biotin and the second binding member comprises streptavidin.

25. The method of claim 23, wherein the first binding member comprises streptavidin and the second binding member comprises biotin.

26. A method of selecting cells, the method comprising:
a) providing in a reaction chamber (i) a plurality of polymer precursors and (ii) a plurality of binding agents capable of binding a subset of a plurality of cells;
b) subjecting the reaction chamber to polymerization conditions to generate, in the reaction chamber, a 3-dimensional (3D) matrix comprising the plurality of binding agents;
c) introducing the plurality of cells into the reaction chamber to allow the plurality of binding agents to bind the subset of the plurality of cells, thereby generating (i) at least one bound cell, and (ii) at least one unbound cells;
d) washing the 3D matrix to remove the at least one unbound cells; and
e) subjecting the 3D matrix to a dissolving reagent to dissolve the 3D matrix, thereby releasing the subset of the plurality of cells.

27. The method of claim 26, wherein the reaction chamber is in a microfluidic device.

28. The method of claim 26, wherein b) comprises contacting the polymer precursors with a polymerization reagent.

29. The method of claim 28, wherein the polymer precursors comprise alginate and the polymerization agent comprises calcium ions or salts.

30. The method of claim 26, wherein the plurality of binding agents comprise antibodies or derivatives thereof.

31. The method of claim 30, wherein the antibodies or derivatives thereof comprise scFvs, nanobodies, or Fab domains.

32. The method of claim 26, wherein the dissolving reagent comprises citrate, EDTA, or alginase.

33. A method of selecting cells, the method comprising:
a) providing a plurality of magnetic beads in a reaction chamber, wherein at least one magnetic bead of the plurality of magnetic beads comprises a binding agent capable of binding a subset of a plurality of cells;
b) introducing the plurality of cells into the reaction chamber to allow the plurality of magnetic beads to bind the subset of the plurality of cells, thereby generating (i) at least one magnetic bead-cell complex and (ii) at least one unbound cell;
c) subjecting (i) the at least one magnetic bead-cell complex and (ii) the at least one unbound cells to a magnetic field to separate the at least one magnetic bead-cell complex from the at least one unbound cell; and
d) subjecting the at least one magnetic bead-cell complex to a dissolving reagent to dissolve the at least one magnetic beads, thereby releasing the subset of a plurality of cells.

34. The method of claim 33, wherein the at least one magnetic bead comprises a plurality of paramagnetic nanoparticles, wherein the plurality of paramagnetic nanoparticles are released during d) to yield released paramagnetic nanoparticles.

35. The method of claim 34, further comprising harvesting the released paramagnetic nanoparticles with a magnet.

36. The method of claim 33, wherein the plurality of magnetic beads comprises alginate.

37. The method of claim 36, wherein the dissolving agent comprises at least one of citric acid, EDTA, and alginase.

38. The method of claim 33, wherein the binding agents comprises an antibody or derivatives thereof.

39. The method of claim 38, wherein the antibody of derivatives thereof comprise antibody fragments.

40. The method of claim 38, wherein the antibodies or derivatives thereof comprise scFvs, nanobodies, or Fab domains.

41. A method of manufacture of dissolvable magnetic beads comprising
a) loading a microfluidic droplet generator with (i) a liquid alginate solution comprising magnetic nanoparticles, and (ii) a mineral oil, into a microchannel; and
b) subjecting the liquid alginate solution to crosslinking, thereby forming dissolvable magnetic beads.

42. The method of claim 41, further comprising loading the microfluidic droplet generator with a binding agent in a), wherein the dissolvable magnetic beads comprise the binding agent.

43. A method of manufacture of dissolvable magnetic beads comprising
a) spray drying a solution of alginate mixed with magnetic nanoparticles; and
b) reconstituting the spray-dried alginate-magnetic nanoparticles in a buffer solution.

44. The method of claim 43, wherein the solution further comprises a plurality of binding agents, and wherein the dissolvable magnetic beads comprise the plurality of binding agents.

45. The method of claim 43, wherein the plurality of binding agents comprise an antibody or a derivative thereof.

46. A method of selecting cells, the method comprising:
a) providing a plurality of beads in a reaction chamber, wherein at least one bead of the plurality of beads comprises a binding agent capable of binding a subset of a plurality of cells;
b) introducing the plurality of cells into the reaction chamber to allow the plurality of beads to bind the subset of the plurality of cells, thereby generating (i) at least one bead-cell complexes, and (ii) at least one unbound cell;
c) subjecting (i) at least one bead-cell complex, and (ii) at least one unbound cell to a separation to separate (i) the plurality of bead-cell complexes from (ii) the plurality of unbound cells; and
d) subjecting the bead-cell complexes to a dissolving reagent to dissolve the plurality of beads, thereby releasing the subset of a plurality of cells.

47. The method of claim 46, wherein the binding agent comprises an antibody.

48. The method of claim 46, wherein the plurality of beads comprise alginate.

49. The method of claim 48, wherein the dissolving reagent comprises citrate.

50. The method of claim 46, wherein the size selection comprises use of a filter.

51. A method of selecting cells, the method comprising:
a) providing, in a chamber, a plurality of cells and a plurality of buoyant beads, wherein at least one buoyant bead of the plurality of buoyant beads comprises a binding agent capable of binding a subset of the plurality of cells, wherein the chamber comprises: a plunger configured to pressurize liquid in the chamber, and the chamber is connected to an input channel and an output channel that is separated from the chamber with a filter.
b) subjecting the plurality of cells and plurality of buoyant beads to conditions to allow the plurality of buoyant beads to bind the subset of the plurality of cells, thereby generating i) at least one buoyant bead-cell complex, and ii) a plurality of unbound cells, wherein the at least one buoyant bead-cell complex is a larger size than a pore of the filter and at least a subset of the plurality of unbound cells is smaller than the pore of the filter;
c) initiating the plunger to push the at least one buoyant bead-cell complex and the plurality of unbound cells towards the filter, wherein the at least a subset of the plurality of unbound cells are able to traverse through the filter and the at least one buoyant bead-cell complex is unable to traverse through the filter;
d) subjecting the at least one buoyant bead-cell complex to a release condition, thereby releasing the subset of the plurality of cells from the buoyant beads; and
e) initiating the plunger to push the subset of plurality of cells towards the filter, wherein the subset of plurality of cells are able to traverse through the filter and exit the chamber.

52. The method of claim 51, wherein initiating the plunger comprises modulating the plunger between push and pull conditions.

53. The method of claim 51, wherein the filter comprises a sieve.

54. The method of claim 51, wherein the binding agent comprises an antibody.

55. The method of claim 51, wherein a buoyant bead of the plurality of the buoyant bead comprises streptavidin.

56. The method of claim 55, wherein the binding agent comprises a biotin and is linked to the buoyant bead via a biotin-streptavidin interaction.

57. The method of claim 51, wherein release condition comprises flowing a solution comprising biotin.

58. The method of claim 51, wherein release condition comprises flowing a solution comprising a high salt concentration.

59. The method of claim 51, wherein release condition comprises flowing a solution comprising a pH lower than the pH of a solution in reaction chamber.

60. The method of claim 51, wherein release condition comprises flowing a solution comprising a pH lower than the pH of a solution in reaction chamber.

61. The method of claim 51, wherein the binding agent can bind a CD3, CD4, or CD8 protein.

62. The method of claim 51, wherein the binding agent can bind T-cells.

63. A system for selecting cells, the system comprising:
a) a chamber comprising a plurality of buoyant beads;
b) an input channel fluidically connected to the chamber;
c) an output channel fluidically connected to the chamber;
d) a filter disposed at an entrance of the output channel from the chamber, and
e) a plunger disposed in the chamber and configured to apply pressure to a fluid in the chamber and push the fluid through the filter into the output channel.

64. The system of claim 63, wherein a buoyant bead of the plurality of buoyant beads comprise a binding agent.

65. The system of claim 64, wherein the binding agent comprises an antibody.

66. The system of claim 63, wherein a buoyant bead of the plurality of the buoyant bead comprises streptavidin.

67. The system of claim 64, wherein the binding agent comprises a biotin and is linked to the buoyant bead via a biotin-streptavidin interaction.

68. The system of claim 64, wherein the binding agent can bind a CD3, CD4, or CD8 protein.

69. The system of claim 64, wherein the binding agent can bind T-cells.
